(19) United States
(12) Patent Application Publication
(10) Pub. No.: US 2015/0246093 A1
(43) Pub. Date: Sep. 3, 2015

(54) ORAL FORMULATIONS OF ANGIOTENSIN

(71) Applicant: TARIX PHARMACEUTICALS LTD., Cambridge, MA (US)

(72) Inventors: Richard Franklin, Cambridge, MA (US); William Stern, Boonton, NJ (US); Austin Vryhof, Boonton, NJ (US)

(21) Appl. No.: 14/428,746

(22) PCT Filed: Sep. 17, 2013
(86) PCT No.: PCT/US2013/060139

§ 371 Date: Mar. 17, 2015

Related U.S. Application Data
(60) Provisional application No. 61/701,972, filed on Sep. 17, 2012.

(51) Int. Cl. A61K 38/08; A61K 47/48; A61K 38/12; A61K 9/48; A61K 31/401

(52) U.S. Cl. CPC A61K 38/085; A61K 9/4858; A61K 9/4891; A61K 31/401; A61K 38/12; A61K 47/48215

(57) ABSTRACT

The present invention provides various formulations for oral delivery of angiotensin peptides.

### CROSS REFERENCE TO RELATED APPLICATIONS

This application claims priority from U.S. Provisional Patent Application Ser. No. 61/701,972, filed on Sep. 17, 2012, the disclosure of which is hereby incorporated in its entirety.

### BACKGROUND

Oral delivery is typically a desired route of administration because it is more convenient and involves less patient discomfort as compared to injection, nasal administration and other administration routes. Oral administration of peptides, however, is generally difficult because peptides are susceptible to degradation. Oral administration of short peptides like angiotensins tends to be even more problematic because short peptides typically lack secondary or tertiary structures and therefore are more susceptible to proteolytic enzymes of both the stomach and intestines. These enzymes can quickly degrade a short peptide, rendering it inactive before it can be absorbed into the bloodstream.

### SUMMARY OF THE INVENTION

The present invention provides compositions and methods for effective oral delivery of an angiotensin peptide. In particular, the present invention provides various oral formulations that preserve stability of an angiotensin peptide and enhance its absorption to the blood stream. As a result, an angiotensin peptide delivered according to the present invention may achieve extended half-life and therapeutically effective bioavailability.

In one aspect, the present invention provides a solid dosage form for oral administration including (a) an angiotensin (1-7) peptide, (b) at least one pharmaceutically acceptable pH-lowering agent, (c) at least one absorption enhancer effective to promote bioavailability of the angiotensin (1-7) peptide, and (d) a protective vehicle.

In some embodiments, a suitable solid dosage form is a capsule or tablet.

In some embodiments, a suitable pH-lowering agent is citric acid. In some embodiments, the citric acid is present in an amount greater than about 200 mg (e.g., greater than about 250 mg, 300 mg, 350 mg, 400 mg, 450 mg, 500 mg, 550 mg, 600 mg, 650 mg, 700 mg, 750 mg, 800 mg). In some embodiments, the citric acid is present in an amount greater than about 20% (e.g., greater than 25%, 30%, 35%, 40%, 45%, 50%, 55%, 60%, 65%, 70%, 75%, 80%) of the total weight of the solid dosage form.

In some embodiments, a suitable pH-lowering agent is tartaric acid.

In some embodiments, a suitable absorption enhancer is an acylcarnitine. In some embodiments, the acylcarnitine is lauroyl carnitine. In some embodiments, the lauroyl carnitine is present in an mount ranging from about 20-200 mg (e.g., ranging from 20-150 mg, 20-100 mg, 20-90 mg, 20-80 mg, 50-200 mg, 50-150 mg, 50-100 mg, 50-90 mg, 50-80 mg). In some embodiments, the lauroyl carnitine is present in an amount of approximately 20 mg, 30 mg, 40 mg, 50 mg, 60 mg, 70 mg, 80 mg, 90 mg, 100 mg, 110 mg, 120 mg, 130 mg, 140 mg, 150 mg, 160 mg, 170 mg, 180 mg, 190 mg, or 200 mg. In some embodiments, the lauroyl carnitine is present in an mount ranging from about 2-20% (e.g., 2-15%, 2-10%, 2-7.5%, 5-20%, 5-15%, 5-10%, 5-7.5%) of the total weight of the solid dosage form. In some embodiments, the lauroyl carnitine is present in an amount of or greater than approximately 1%, 2%, 3%, 4%, 5%, 6%, 7%, 8%, 9%, 10%, 11%, 12%, 13%, 14%, 15%, 16%, 17%, 18%, 19% or 20% of the total weight of the solid dosage form. In some embodiments, the lauroyl carnitine is present in an amount of or less than approximately 25%, 24%, 23%, 22%, 21%, 20%, 19%, 18%, 17%, 16%, 15%, 14%, 13%, 12%, 11%, 10%, 9%, 8%, 7%, 6%, 5% of the total weight of the solid dosage form.

In some embodiments, a suitable protective vehicle is an enteric coat. In some embodiments, the protective vehicle constitutes an amount of or less than approximately 25%, 24%, 23%, 22%, 21%, 20%, 19%, 18%, 17%, 16%, 15%, 14%, 13%, 12%, 11%, 10%, 9%, 8%, 7%, 6%, 5% of the total weight of the solid dosage form.

In some embodiments, a solid dosage form according to the present invention further comprises one or more excipients. In particular embodiments, the one or more excipients are selected from fillers such as PROSOLV®, disintegrants such as POLYPLASDONE™ crospovidone, glidants such as silicon dioxide or lubricants such as sodium stearyl fumarate.

In some embodiments, a solid dosage form according to the invention further comprises captopril.

In some embodiments, a suitable solid dosage form has a total weight ranging from about 500-1500 (e.g., from about 500-1200 mg, 500-1000 mg, 600-1500 mg, 600-1200 mg, 600-1000 mg, 700-1500 mg, 700-1200 mg, 700-1000 mg, 800-1500 mg, 800-1200 mg, 800-1000 mg). In some embodiments, a suitable solid dosage form has a total weight of or greater than about 500 mg, 600 mg, 700 mg, 800 mg, 900 mg, 1000 mg, 1100 mg, 1200 mg, 1300 mg, 1400 mg, or 1500 mg. In some embodiments, a suitable solid dosage form has a total weight of or less than about 2000 mg, 1900 mg, 1800 mg, 1700 mg, 1600 mg, 1500 mg, 1400 mg, 1300 mg, 1200 mg, 1100 mg, 1000 mg, 900 mg, 800 mg, 700 mg, 600 mg, or 500 mg.

In some embodiments, an angiotensin (1-7) peptide is present in an amount ranging from about 10-1000 mg (e.g., about 10-900 mg, 10-800 mg, 10-700 mg, 10-600 mg, 10-500 mg, 100-1000 mg, 100-900 mg, 100-800 mg, 100-700 mg, 100-600 mg, 100-500 mg, 100-400 mg, 100-300 mg, 200-1000 mg, 200-900 mg, 200-800 mg, 200-700 mg, 200-600 mg, 200-500 mg, 200-400 mg, 300-1000 mg, 300-900 mg, 300-800 mg, 300-700 mg, 300-600 mg, 300-500 mg). In some embodiments, an angiotensin (1-7) peptide is present in an amount of or greater than about 10 mg, 50 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg. In some embodiments, an angiotensin (1-7) peptide is present in an amount of or less than about 1000 mg, 950 mg, 900 mg, 850 mg, 800 mg, 750 mg, 700 mg, 650 mg, 600 mg, 550 mg, 500 mg, 450 mg, 400 mg, 350 mg, 300 mg, 250 mg, 200 mg, 150 mg, or 100 mg.

In particular embodiments, present invention provides a solid dosage form for oral administration including (a) an angiotensin (1-7) peptide, (b) citric acid, (c) lauroyl carnitine, and (d) a protective vehicle. In certain embodiments, the citric acid is present in an amount great than 500 mg and the lauroyl carnitine is present in an amount ranging from 50-100 mg.

In certain embodiments, the solid dosage form is a capsule or tablet. In certain embodiments, a suitable protective vehicle is an enteric coat.

In various embodiments, an angiotensin (1-7) peptide comprises the naturally-occurring Angiotensin (1-7) amino acid sequence of Asp¹-Arg²-Val³-Tyr⁴-Ile⁵-His⁶-Pro⁷ (SEQ ID NO:1).

In various embodiments, an angiotensin (1-7) peptide is a functional equivalent of SEQ ID NO:1. In some embodiments, the functional equivalent is a linear peptide. In some embodiments, the linear peptide comprises a sequence that includes at least four amino acids from the seven amino acids that appear in the naturally-occurring Angiotensin (1-7), wherein the at least four amino acids maintain their relative positions as they appear in the naturally-occurring Angiotensin (1-7). In some embodiments, the linear peptide comprises a sequence that includes at least five amino acids from the seven amino acids that appear in the naturally-occurring Angiotensin (1-7), wherein the at least five amino acids maintain their relative positions as they appear in the naturally-occurring Angiotensin (1-7). In some embodiments, the linear peptide comprises a sequence that includes at least six amino acids from the seven amino acids that appear in the naturally-occurring Angiotensin (1-7), wherein the at least six amino acids maintain their relative positions as they appear in the naturally-occurring Angiotensin (1-7). In some embodiments, the at least four, five or six amino acids, respectively, further maintain their relative spacing as they appear in the naturally-occurring Angiotensin (1-7).

In some embodiments, the linear peptide contains 4-25 amino acids (e.g., 4-20, 4-15, 4-14, 4-13, 4-12, 4-11, 4-10, 4-9, 4-8, 4-7 amino acids). In some embodiments, the linear peptide contains 4, 5, 6, 7, 8, 9, 10, 11, 12, 13, 14, 16, 17, 18, 19, 20, 21, 22, 23, 24, or 25 amino acids.

In some embodiments, the linear peptide is a fragment of the naturally-occurring Angiotensin (1-7).

In some embodiments, the linear peptide contains amino acid substitutions, deletions and/or insertions in the naturally-occurring Angiotensin (1-7).

In particular embodiments, the linear peptide has an amino acid sequence of Asp¹-Arg²-Nle³-Tyr⁴-Ile⁵-His⁶-Pro⁷ (SEQ ID NO:2).

In particular embodiments, the linear peptide has an amino acid sequence of Asp¹-Arg²-Val³-Ser⁴-Ile⁵-His⁶-Cys⁷ (SEQ ID NO:6).

In some embodiments, the functional equivalent is a cyclic peptide. In some embodiments, the cyclic peptide comprises a linkage between amino acids. In some embodiments, the linkage is located at residues corresponding to positions Tyr⁴ and Pro⁷ in naturally-occurring Angiotensin (1-7). In some embodiments, the linkage is a thioether bridge.

In particular embodiments, the cyclic peptide comprises an amino acid sequence otherwise identical to the naturally-occurring Angiotensin (1-7) amino acid sequence of Asp¹-Arg²-Val³-Tyr⁴-Ile⁵-His⁶-Pro⁷ (SEQ ID NO:1).

In certain embodiments, the cyclic peptide comprises a norleucine (Nle) replacing position Val³ in naturally-occurring Angiotensin (1-7).

In certain embodiments, the cyclic peptide is a 4,7-cyclized angiotensin (1-7) with the following formula:

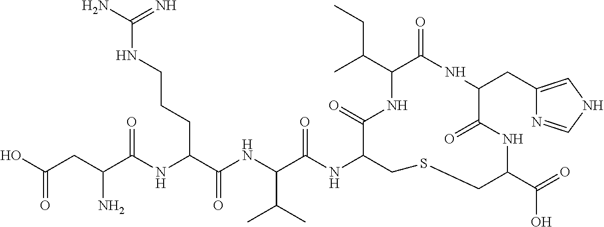 [SMILES: CCC(C)C1NC(=O)C(NC(=O)C(NC(=O)C(CCCNC(=N)N)NC(=O)C(N)CC(=O)O)C(C)C)CSCC(C(=O)O)NC(=O)C(Cc2c[nH]cn2)NC1=O]

In various embodiments, the angiotensin (1-7) peptide comprises one or more chemical modifications to increase protease resistance, serum stability and/or bioavailability. In some embodiments, the one or more chemical modifications comprise pegylation.

The present invention further provides methods for administering an oral formulation described herein.

As used in this application, the terms “about” and “approximately” are used as equivalents. Any numerals used in this application with or without about/approximately are meant to cover any normal fluctuations appreciated by one of ordinary skill in the relevant art.

Other features, objects, and advantages of the present invention are apparent in the detailed description that follows. It should be understood, however, that the detailed description, while indicating embodiments of the present invention, is given by way of illustration only, not limitation. Various changes and modifications within the scope of the invention will become apparent to those skilled in the art from the detailed description.

### BRIEF DESCRIPTION OF THE DRAWING

The drawing is for illustration purposes only, not for limitation.

FIG. 1 depicts exemplary results illustrating total exposure of angiotensin (1-7) represented by area under the curve (AUC) compared between the various routes of administration.

### DEFINITIONS

In order for the present invention to be more readily understood, certain terms are first defined below. Additional definitions for the following terms and other terms are set forth throughout the specification.

Animal: As used herein, the term “animal” refers to any member of the animal kingdom. In some embodiments, “animal” refers to humans, at any stage of development. In some embodiments, “animal” refers to non-human animals, at any stage of development. In certain embodiments, the non-human animal is a mammal (e.g., a rodent, a mouse, a rat, a rabbit, a monkey, a dog, a cat, a sheep, cattle, a primate, and/or a pig). In some embodiments, animals include, but are not limited to, mammals, birds, reptiles, amphibians, fish, insects, and/or worms. In some embodiments, an animal may be a transgenic animal, genetically-engineered animal, and/or a clone.

Approximately or about: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Bioavailability: As used herein, the term “bioavailability” generally refers to the percentage of the administered dose that reaches the blood stream of a subject.

Biologically active: As used herein, the phrase “biologically active” refers to a characteristic of any agent that has activity in a biological system, and particularly in an organism. For instance, an agent that, when administered to an organism, has a biological effect on that organism, is considered to be biologically active. In particular embodiments, where a peptide is biologically active, a portion of that peptide that shares at least one biological activity of the peptide is typically referred to as a “biologically active” portion. In certain embodiments, a peptide has no intrinsic biological activity but that inhibits the effects of one or more naturally-occurring angiotensin compounds is considered to be biologically active.

Carrier or diluent: As used herein, the terms “carrier” and “diluent” refers to a pharmaceutically acceptable (e.g., safe and non-toxic for administration to a human) carrier or diluting substance useful for the preparation of a pharmaceutical formulation. Exemplary diluents include sterile water, bacteriostatic water for injection (BWFI), a pH buffered solution (e.g. phosphate-buffered saline), sterile saline solution, Ringer's solution or dextrose solution.

Dosage form: As used herein, the terms “dosage form” and “unit dosage form” refer to a physically discrete unit of a therapeutic agent for the patient to be treated. Each unit contains a predetermined quantity of active material calculated to produce the desired therapeutic effect. It will be understood, however, that the total dosage of the composition will be decided by the attending physician within the scope of sound medical judgment.

Dosing regimen: A “dosing regimen” (or “therapeutic regimen”), as that term is used herein, is a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which are separated from one another by a time period of the same length; in some embodiments, a dosing regime comprises a plurality of doses and at least two different time periods separating individual doses. In some embodiments, the therapeutic agent is administered continuously over a predetermined period. In some embodiments, the therapeutic agent is administered once a day (QD) or twice a day (BID).

Excipient: As used herein, the term “excipient” refers to any inert substance added to a drug and/or formulation for the purposes of improving its physical qualities (i.e. consistency), pharmacokinetic properties (i.e. bioavailability), pharmacodynamic properties and combinations thereof.

Functional equivalent or functional derivative: As used herein, the term “functional equivalent” or “functional derivative” denotes, in the context of a functional derivative of an amino acid sequence, a molecule that retains a biological activity that is substantially similar to that of the original sequence. A functional derivative or equivalent may be a natural derivative or is prepared recombinantly or synthetically. Exemplary functional derivatives include amino acid sequences having substitutions, deletions, or additions of one or more amino acids, provided that the biological activity of the protein is conserved. The substituting amino acid desirably has chemico-physical properties which are similar to that of the substituted amino acid. Desirable similar chemico-physical properties include, similarities in charge, bulkiness, hydrophobicity, hydrophilicity, and the like.

Improve, increase, or reduce: As used herein, the terms “improve,” “increase” or “reduce,” or grammatical equivalents, indicate values that are relative to a baseline measurement, such as a measurement in the same individual prior to initiation of the treatment described herein, or a measurement in a control individual (or multiple control individuals) in the absence of the treatment described herein. A “control individual” is an individual afflicted with the same form of disease as the individual being treated, who is about the same age as the individual being treated (to ensure that the stages of the disease in the treated individual and the control individual(s) are comparable).

In vitro: As used herein, the term “in vitro” refers to events that occur in an artificial environment, e.g., in a test tube or reaction vessel, in cell culture, etc., rather than within a multi-cellular organism.

In vivo: As used herein, the term “in vivo” refers to events that occur within a multi-cellular organism, such as a human and a non-human animal. In the context of cell-based systems, the term may be used to refer to events that occur within a living cell (as opposed to, for example, in vitro systems).

Isolated: As used herein, the term “isolated” refers to a substance and/or entity that has been (1) separated from at least some of the components with which it was associated when initially produced (whether in nature and/or in an experimental setting), and/or (2) produced, prepared, and/or manufactured by the hand of man. Isolated substances and/or entities may be separated from at least about 10%, about 20%, about 30%, about 40%, about 50%, about 60%, about 70%, about 80%, about 90%, about 95%, about 98%, about 99%, substantially 100%, or 100% of the other components with which they were initially associated. In some embodiments, isolated agents are more than about 80%, about 85%, about 90%, about 91%, about 92%, about 93%, about 94%, about 95%, about 96%, about 97%, about 98%, about 99%, substantially 100%, or 100% pure. As used herein, a substance is “pure” if it is substantially free of other components. As used herein, the term “isolated cell” refers to a cell not contained in a multi-cellular organism.

Peptide: The term “peptide” as used herein refers a sequential chain of amino acids linked together via peptide bonds. Typically, the term is used to refer to an amino acid chain of short length, but one of ordinary skill in the art will understand that the term is not limited to any particular length chains and can refer to a minimal chain comprising two amino acids linked together via a peptide bond. Typically, however, a peptide refers to an amino acid chain of or less than 50, 45, 40, 35, 30, 25, 20, 15, 10 amino acids. As is known to those skilled in the art, peptides may be processed and/or modified.

Pharmaceutically acceptable: As used herein, the term “pharmaceutically-acceptable” refers to any entity or composition that does not produce an undesirable allergic or antigenic response when administered to a subject.

Protein: The term “protein” as used herein refers to one or more polypeptides that function as a discrete unit. If a single polypeptide is the discrete functioning unit and does not require permanent or temporary physical association with other polypeptides in order to form the discrete functioning unit, the terms “polypeptide” and “protein” may be used interchangeably. If the discrete functional unit is comprised of more than one polypeptide that physically associate with one another, the term “protein” refers to the multiple polypeptides that are physically coupled and function together as the discrete unit.

Stability: As used herein, the term “stable” refers to the ability of the therapeutic agent to maintain its therapeutic efficacy (e.g., all or the majority of its intended biological activity and/or physiochemical integrity) over extended periods of time. The stability of a therapeutic agent, and the capability of the pharmaceutical composition to maintain stability of such therapeutic agent, may be assessed over extended periods of time (e.g., for at least 1, 3, 6, 12, 18, 24, 30, 36 months or more). In certain embodiments, pharmaceutical compositions described herein have been formulated such that they are capable of stabilizing, or alternatively slowing or preventing the degradation, of one or more therapeutic agents formulated therewith. In the context of a formulation a stable formulation is one in which the therapeutic agent therein essentially retains its physical and/or chemical integrity and biological activity upon storage and during processes (such as freeze/thaw, mechanical mixing and lyophilization).

Subject: As used herein, the term “subject” refers to a human or any non-human animal (e.g., mouse, rat, rabbit, dog, cat, cattle, swine, sheep, horse or primate). A human includes pre and post natal forms. In many embodiments, a subject is a human being. A subject can be a patient, which refers to a human presenting to a medical provider for diagnosis or treatment of a disease. The term “subject” is used herein interchangeably with “individual” or “patient.” A subject can be afflicted with or is susceptible to a disease or disorder but may or may not display symptoms of the disease or disorder.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Therapeutically effective amount: As used herein, the term “therapeutically effective amount” of a therapeutic agent means an amount that is sufficient, when administered to a subject suffering from or susceptible to a disease, disorder, and/or condition, to treat, diagnose, prevent, and/or delay the onset of the symptom(s) of the disease, disorder, and/or condition. It will be appreciated by those of ordinary skill in the art that a therapeutically effective amount is typically administered via a dosing regimen comprising at least one unit dose.

Treating: As used herein, the term “treat,” “treatment,” or “treating” refers to any method used to partially or completely alleviate, ameliorate, relieve, inhibit, prevent, delay onset of, reduce severity of and/or reduce incidence of one or more symptoms or features of a particular disease, disorder, and/or condition. Treatment may be administered to a subject who does not exhibit signs of a disease and/or exhibits only early signs of the disease for the purpose of decreasing the risk of developing pathology associated with the disease.

Other features, objects, and advantages of the present invention are apparent in the detailed description that follows. It should be understood, however, that the detailed description, while indicating embodiments of the present invention, is given by way of illustration only, not limitation. Various changes and modifications within the scope of the invention will become apparent to those skilled in the art from the detailed description.

### DETAILED DESCRIPTION OF CERTAIN EMBODIMENTS

Among other things, the present invention provides formulations of angiotensin (1-7) (Ang-(1-7)) suitable for oral administration to a subject. Such administration could be for a variety of reasons including treatment of a disease, disorder or condition.

In some embodiments, a solid dosage form for oral administration is provided including (a) an angiotensin (1-7) peptide, (b) at least one pharmaceutically acceptable pH-lowering agent, (c) at least one absorption enhancer effective to promote bioavailability of the angiotensin (1-7) peptide, and (d) a protective vehicle.

In some embodiments, the solid dosage form is a capsule or tablet. Various methods and ingredients for making oral formulations are known in the art and it is expected that one of skill would be able to determine which of these methods and ingredients will be compatible with the invention as described in this specification. Such methods and ingredients are also contemplated as within the scope of the present invention.

Various aspects of the invention are described in detail in the following sections. The use of sections is not meant to limit the invention. Each section can apply to any aspect of the invention. In this application, the use of “or” means “and/or” unless stated otherwise.

### Angiotensin (1-7) Peptides

As used herein, the term “angiotensin (1-7) peptide” refers to both naturally-occurring Angiotensin (1-7) and any functional equivalent, analogue or derivative of naturally-occurring Angiotensin (1-7). As used herein, “peptide” and “polypeptide” are interchangeable terms and refer to two or more amino acids bound together by a peptide bond. As used herein, the terms “peptide” and “polypeptide” include both linear and cyclic peptide. The terms “angiotensin-(1-7)”, “Angiotensin-(1-7)”, and “Ang-(1-7)” are used interchangeably.

Naturally-Occurring Angiotensin (1-7)

Naturally-occurring Angiotensin (1-7) (also referred to as Ang-(1-7)) is a seven amino acid peptide shown below:

(SEQ ID NO: 1)
Asp¹-Arg²-Val³-Tyr⁴-Ile⁵-His⁶-Pro⁷

It is part of the renin-angiotensin system and is converted from a precursor, also known as Angiotensinogen, which is an α-2-globulin that is produced constitutively and released into the circulation mainly by the liver. Angiotensinogen is a member of the serpin family and also known as renin substrate. Human angiotensinogen is 452 amino acids long, but other species have angiotensinogen of varying sizes. Typically, the first 12 amino acids are the most important for angiotensin activity:

(SEQ ID NO: 4)
Asp¹-Arg²-Val³-Tyr⁴-Ile⁵-His⁶-Pro⁷-Phe⁸-His⁹-Leu¹⁰-
Val¹¹-Ile¹²

Different types of angiotensin may be formed by the action of various enzymes. For example, Angiotensin (1-7) is generated by action of Angiotensin-converting enzyme 2 (ACE 2).

Ang-(1-7) is an endogenous ligand for Mas receptors. Mas receptors are G-protein coupled receptor containing seven transmembrane spanning regions. As used herein, the term “angiotensin-(1-7) receptor′ encompasses the G Protein-Coupled Mas Receptors.

As used herein, the term “naturally-occurring Angiotensin (1-7)” includes any Angiotensin (1-7) peptide purified from natural sources and any recombinantly produced or chemically synthesized peptides that have an amino acid sequence identical to that of the naturally-occurring Angiotensin (1-7).

Functional Equivalents, Analogs or Derivatives of Ang-(I-7)

In some embodiments, an angiotensin (1-7) peptide suitable for the present invention is a functional equivalent of naturally-occurring Ang-(1-7). As used herein, a functional equivalent of naturally-occurring Ang-(1-7) refers to any peptide that shares amino acid sequence identity to the naturally-occurring Ang-(1-7) and retain substantially the same or similar activity as the naturally-occurring Ang-(1-7). For example, in some embodiments, a functional equivalent of naturally-occurring Ang-(1-7) described herein has pro-angiogenic activity as determined using methods described herein or known in the art, or an activity such as nitric oxide release, vasodilation, improved endothelial function, antidiuresis, or one of the other properties discussed herein, that positively impacts angiogenesis. In some embodiments, a functional equivalent of naturally-occurring Ang-(1-7) described herein can bind to or activate an angiotensin-(1-7) receptor (e.g., the G protein-coupled Mas receptor) as determined using various assays described herein or known in the art. In some embodiments, a functional equivalent of Ang-(1-7) is also referred to as an angiotensin (1-7) analogue or derivative, or functional derivative.

Typically, a functional equivalent of angiotensin (1-7) shares amino acid sequence similarity to the naturally-occurring Ang-(1-7). In some embodiments, a functional equivalent of Ang-(1-7) according to the invention contains a sequence that includes at least 3 (e.g., at least 4, at least 5, at least 6, at least 7) amino acids from the seven amino acids that appear in the naturally-occurring Ang-(1-7), wherein the at least 3 (e.g., at least 4, at least 5, at least 6, or at least 7) amino acids maintain their relative positions and/or spacing as they appear in the naturally-occurring Ang-(1-7).

In some embodiments, a functional equivalent of Ang-(1-7) also encompass any peptide that contain a sequence at least 50% (e.g., at least 60%, 70%, 80%, or 90%) identical to the amino acid sequence of naturally-occurring Ang-(1-7). Percentage of amino acid sequence identity can be determined by alignment of amino acid sequences. Alignment of amino acid sequences can be achieved in various ways that are within the skill in the art, for instance, using publicly available computer software such as BLAST, ALIGN or Megalign (DNASTAR) software. Those skilled in the art can determine appropriate parameters for measuring alignment, including any algorithms needed to achieve maximal alignment over the full length of the sequences being compared. Preferably, the WU-BLAST-2 software is used to determine amino acid sequence identity (Altschul et al., Methods in Enzymology 266, 460-480 (1996); http://blast.wustl/edu/blast/README.html). WU-BLAST-2 uses several search parameters, most of which are set to the default values. The adjustable parameters are set with the following values: overlap span=1, overlap fraction=0.125, word threshold (T)=11. HSP score (S) and HSP S2 parameters are dynamic values and are established by the program itself, depending upon the composition of the particular sequence, however, the minimum values may be adjusted and are set as indicated above.

In some embodiments, a functional equivalent, analogue or derivative of Ang-(1-7) is a fragment of the naturally-occurring Ang-(1-7). In some embodiments, a functional equivalent, analogue or derivative of Ang-(1-7) contains amino acid substitutions, deletions and/or insertions in the naturally-occurring Ang-(1-7). Ang-(1-7) functional equivalents, analogues or derivatives can be made by altering the amino acid sequences by substitutions, additions, and/or deletions. For example, one or more amino acid residues within the sequence of the naturally-occurring Ang-(1-7) (SEQ ID NO:1) can be substituted by another amino acid of a similar polarity, which acts as a functional equivalent, resulting in a silent alteration. Substitution for an amino acid within the sequence may be selected from other members of the class to which the amino acid belongs. For example, the positively charged (basic) amino acids include arginine, lysine, and histidine. The nonpolar (hydrophobic) amino acids include leucine, isoleucine, alanine, phenylalanine, valine, proline, tryptophane, and methionine. The uncharged polar amino acids include serine, threonine, cysteine, tyrosine, asparagine, and glutamine. The negatively charged (acid) amino acids include glutamic acid and aspartic acid. The amino acid glycine may be included in either the nonpolar amino acid family or the uncharged (neutral) polar amino acid family. Substitutions made within a family of amino acids are generally understood to be conservative substitutions. For example, the amino acid sequence of a peptide inhibitor can be modified or substituted.

Examples of Ang-(1-7) functional equivalents, analogues and derivatives are described in the section entitled “Exemplary Angiotensin(1-7) Peptides” below.

An angiotensin-(1-7) peptide can be of any length. In some embodiments, an angiotensin-(1-7) peptide according to the present invention can contain, for example, from 4-25 amino acids (e.g., 4-20, 4-15, 4-14, 4-13, 4-12, 4-11, 4-10, 4-9, 4-8, 4-7 amino acids). In some embodiments, the linear peptide contains 4, 5, 6, 7, 8, 9, 10, 11, 12, 13, 14, 16, 17, 18, 19, 20, 21, 22, 23, 24, or 25 amino acids.

In some embodiments, an angiotensin-(1-7) peptide contains one or more modifications to increase protease resistance, serum stability and/or bioavailability. In some embodiments, suitable modifications are selected from pegylation, acetylation, glycosylation, biotinylation, substitution with D-amino acid and/or un-natural amino acid, and/or cyclization of the peptide.

As used herein, the term “amino acid,” in its broadest sense, refers to any compound and/or substance that can be incorporated into a polypeptide chain. In certain embodiments, an amino acid has the general structure H₂N—C(H)(R)—COOH. In certain embodiments, an amino acid is a naturally-occurring amino acid. In certain embodiments, an amino acid is a synthetic or un-natural amino acid (e.g., α,α-disubstituted amino acids, N-alkyl amino acids); in some embodiments, an amino acid is a d-amino acid; in certain embodiments, an amino acid is an 1-amino acid. “Standard amino acid” refers to any of the twenty standard amino acids commonly found in naturally occurring peptides including both 1- and d-amino acids which are both incorporated in peptides in nature. “Nonstandard” or “unconventional amino acid” refers to any amino acid, other than the standard amino acids, regardless of whether it is prepared synthetically or obtained from a natural source. As used herein, “synthetic or un-natural amino acid” encompasses chemically modified amino acids, including but not limited to salts, amino acid derivatives (such as amides), and/or substitutions. Amino acids, including carboxy- and/or amino-terminal amino acids in peptides, can be modified by methylation, amidation, acetylation, and/or substitution with other chemical groups that can change the peptide's circulating half-life without adversely affecting its activity. Examples of unconventional or un-natural amino acids include, but are not limited to, citrulline, ornithine, norleucine, norvaline, 4-(E)-butenyl-4(R)-methyl-N-methylthreonine (MeBmt), N-methyl-leucine (MeLeu), aminoisobutyric acid, statine, and N-methyl-alanine (MeAla). Amino acids may participate in a disulfide bond. The term “amino acid” is used interchangeably with “amino acid residue,” and may refer to a free amino acid and/or to an amino acid residue of a peptide. It will be apparent from the context in which the term is used whether it refers to a free amino acid or a residue of a peptide.

In certain embodiments, angiotensin-(1-7) peptides contain one or more L-amino acids, D-amino acids, and/or un-natural amino acids.

In addition to peptides containing only naturally occurring amino acids, peptidomimetics or peptide analogs are also encompassed by the present invention. Peptide analogs are commonly used in the pharmaceutical industry as non-peptide drugs with properties analogous to those of the template peptide. The non-peptide compounds are termed “peptide mimetics” or peptidomimetics (Fauchere et al., Infect. Immun. 54:283-287 (1986); Evans et al., J. Med. Chem. 30:1229-1239 (1987)). Peptide mimetics that are structurally related to therapeutically useful peptides and may be used to produce an equivalent or enhanced therapeutic or prophylactic effect. Generally, peptidomimetics are structurally similar to the paradigm polypeptide (i.e., a polypeptide that has a biological or pharmacological activity) such as naturally-occurring receptor-binding polypeptides, but have one or more peptide linkages optionally replaced by linkages such as —CH₂NH—, —CH₂S—, —CH₂—CH₂—, —CH═CH— (cis and trans), —CH₂SO—, —CH(OH)CH₂—, —COCH₂— etc., by methods well known in the art (Spatola, Peptide Backbone Modifications, Vega Data, 1(3):267 (1983); Spatola et al. Life Sci. 38:1243-1249 (1986); Hudson et al. Int. J. Pept. Res. 14:177-185 (1979); and Weinstein. B., 1983, Chemistry and Biochemistry, of Amino Acids, Peptides and Proteins, Weinstein eds, Marcel Dekker, New-York,). Such peptide mimetics may have significant advantages over naturally-occurring polypeptides including more economical production, greater chemical stability, enhanced pharmacological properties (e.g., half-life, absorption, potency, efficiency, etc.), reduced antigenicity and others.

Ang-(1-7) peptides also include other types of peptide derivatives containing additional chemical moieties not normally part of the peptide, provided that the derivative retains the desired functional activity of the peptide. Examples of such derivatives include (1) N-acyl derivatives of the amino terminal or of another free amino group, wherein the acyl group may be an alkanoyl group (e.g., acetyl, hexanoyl, octanoyl) an aroyl group (e.g., benzoyl) or a blocking group such as F-moc (fluorenylmethyl-O—CO—); (2) esters of the carboxy terminal or of another free carboxy or hydroxyl group; (3) amide of the carboxy-terminal or of another free carboxyl group produced by reaction with ammonia or with a suitable amine; (4) phosphorylated derivatives; (5) derivatives conjugated to an antibody or other biological ligand and other types of derivatives; and (6) derivatives conjugated to a polyethylene glycol (PEG) chain.

Ang-(1-7) peptides may be obtained by any method of peptide synthesis known to those skilled in the art, including synthetic (e.g., exclusive solid phase synthesis, partial solid phase synthesis, fragment condensation, classical solution synthesis, native-chemical ligation) and recombinant techniques. For example, the peptides or peptides derivatives can be obtained by solid phase peptide synthesis, which in brief, consist of coupling the carboxyl group of the C-terminal amino acid to a resin (e.g., benzhydrylamine resin, chloromethylated resin, hydroxymethyl resin) and successively adding N-alpha protected amino acids. The protecting groups may be any such groups known in the art. Before each new amino acid is added to the growing chain, the protecting group of the previous amino acid added to the chain is removed. Such solid phase synthesis has been disclosed, for example, by Merrifield, J. Am. Chem. Soc. 85: 2149 (1964); Vale et al., Science 213:1394-1397 (1981), in U.S. Pat. Nos. 4,305,872 and 4,316, 891, Bodonsky et al. Chem. Ind. (London), 38:1597 (1966); and Pietta and Marshall, Chem. Comm. 650 (1970) by techniques reviewed in Lubell et al. “Peptides” Science of Synthesis 21.11, Chemistry of Amides. Thieme, Stuttgart, 713-809 (2005). The coupling of amino acids to appropriate resins is also well known in the art and has been disclosed in U.S. Pat. No. 4,244,946. (Reviewed in Houver-Weyl, Methods of Organic Chemistry. Vol E22a. Synthesis of Peptides and Peptidomimetics, Murray Goodman, Editor-in-Chief, Thieme. Stuttgart. New York 2002).

Unless defined otherwise, the scientific and technological terms and nomenclature used herein have the same meaning as commonly understood by a person of ordinary skill to which this invention pertains. Generally, the procedures of cell cultures, infection, molecular biology methods and the like are common methods used in the art. Such standard techniques can be found in reference manuals such as, for example, Ausubel et al., Current Protocols in Molecular Biology, Wiley Interscience, New York, 2001; and Sambrook et al., Molecular Cloning: A Laboratory Manual, 3^{rd} edition, Cold Spring Harbor Laboratory Press, N.Y., 2001.

During any process of the preparation of an Ang-(1-7) peptide, it may be desirable to protect sensitive reactive groups on any of the molecule concerned. This may be achieved by means of conventional protecting groups such as those described in Protective Groups In Organic Synthesis by T. W. Greene & P. G. M. Wuts, 1991, John Wiley and Sons, New-York; and Peptides: chemistry and Biology by Sewald and Jakubke, 2002, Wiley-VCH, Wheinheim p.142. For example, alpha amino protecting groups include acyl type protecting groups (e.g., trifluoroacetyl, formyl, acetyl), aliphatic urethane protecting groups (e.g., t-butyloxycarbonyl (BOC), cyclohexyloxycarbonyl), aromatic urethane type protecting groups (e.g., fluorenyl-9-methoxy-carbonyl (Fmoc), benzyloxycarbonyl (Cbz), Cbz derivatives) and alkyl type protecting groups (e.g., triphenyl methyl, benzyl). The amino acids side chain protecting groups include benzyl (for Thr and Ser), Cbz (Tyr, Thr, Ser, Arg, Lys), methyl ethyl, cyclohexyl (Asp, His), Boc (Arg, His, Cys) etc. The protecting groups may be removed at a convenient subsequent stage using methods known in the art.

Further, Ang-(1-7) peptides may be synthesized according to the FMOC protocol in an organic phase with protective groups. Desirably, the peptides are purified with a yield of 70% with high-pressure liquid chromatography (HPLC) on a C18 chromatography column and eluted with an acetonitrile gradient of 10-60%. The molecular weight of a peptide can be verified by mass spectrometry (reviewed in Fields, G. B. “Solid-Phase Peptide Synthesis” Methods in Enzymology. Vol. 289, Academic Press, 1997).

Alternatively, Ang-(1-7) peptides may be prepared in recombinant systems using, for example, polynucleotide sequences encoding the polypeptides. It is understood that a polypeptide may contain more than one of the above-described modifications within the same polypeptide.

While peptides may be effective in eliciting a biological activity in vitro, their effectiveness in vivo might be reduced by the presence of proteases. Serum proteases have specific substrate requirements. The substrate must have both L-amino acids and peptide bonds for cleavage. Furthermore, exopeptidases, which represent the most prominent component of the protease activity in serum, usually act on the first peptide bond of the peptide and require a free N-terminus (Powell et al., Pharm. Res. 10:1268-1273 (1993)). In light of this, it is often advantageous to use modified versions of peptides. The modified peptides retain the structural characteristics of the original L-amino acid peptides that confer the desired biological activity of Ang-(1-7) but are advantageously not readily susceptible to cleavage by protease and/or exopeptidases.

Systematic substitution of one or more amino acids of a consensus sequence with D-amino acid of the same type (e.g., D-lysine in place of L-lysine) may be used to generate more stable peptides. Thus, a peptide derivative or peptidomimetic of the present invention may be all L, all D or mixed D, L peptide, in either forward or reverse order. The presence of an N-terminal or C-terminal D-amino acid increases the in vivo stability of a peptide since peptidases cannot utilize a D-amino acid as a substrate (Powell et al., Pharm. Res. 10:1268-1273 (1993)). Reverse-D peptides are peptides containing D-amino acids, arranged in a reverse sequence relative to a peptide containing L-amino acids. Thus, the C-terminal residue of an L-amino acid peptide becomes N-terminal for the D-amino acid peptide, and so forth. Reverse D-peptides retain the same secondary conformation and therefore similar activity, as the L-amino acid peptides, but are more resistant to enzymatic degradation in vitro and in vivo, and thus can have greater therapeutic efficacy than the original peptide (Brady and Dodson, Nature 368:692-693 (1994); Jameson et al., Nature 368:744-746 (1994)). Similarly, a reverse-L peptide may be generated using standard methods where the C-terminus of the parent peptide becomes takes the place of the N-terminus of the reverse-L peptide. It is contemplated that reverse L-peptides of L-amino acid peptides that do not have significant secondary structure (e.g., short peptides) retain the same spacing and conformation of the side chains of the L-amino acid peptide and therefore often have the similar activity as the original L-amino acid peptide. Moreover, a reverse peptide may contain a combination of L- and D-amino acids. The spacing between amino acids and the conformation of the side chains may be retained resulting in similar activity as the original L-amino acid peptide.

Another effective approach to confer resistance to peptidases acting on the N-terminal or C-terminal residues of a peptide is to add chemical groups at the peptide termini, such that the modified peptide is no longer a substrate for the peptidase. One such chemical modification is glycosylation of the peptides at either or both termini. Certain chemical modifications, in particular N-terminal glycosylation, have been shown to increase the stability of peptides in human serum (Powell et al., Pharm. Res. 10:1268-1273 (1993)). Other chemical modifications which enhance serum stability include, but are not limited to, the addition of an N-terminal alkyl group, consisting of a lower alkyl of from one to twenty carbons, such as an acetyl group, and/or the addition of a C-terminal amide or substituted amide group. In particular, the present invention includes modified peptides consisting of peptides bearing an N-terminal acetyl group and/or a C-terminal amide group.

Substitution of non-naturally-occurring amino acids for natural amino acids in a subsequence of the peptides can also confer resistance to proteolysis. Such a substitution can, for instance, confer resistance to proteolysis by exopeptidases acting on the N-terminus without affecting biological activity. Examples of non-naturally-occurring amino acids include α,α-disubstituted amino acids, N-alkyl amino acids, C-α-methyl amino acids, β-amino acids, and β-methyl amino acids. Amino acids analogs useful in the present invention may include, but are not limited to, β-alanine, norvaline, norleucine, 4-aminobutyric acid, orithine, hydroxyproline, sarcosine, citrulline, cysteic acid, cyclohexylalanine, 2-aminoisobutyric acid, 6-aminohexanoic acid, t-butylglycine, phenylglycine, o-phosphoserine, N-acetyl serine, N-formylmethionine, 3-methylhistidine and other unconventional amino acids. Furthermore, the synthesis of peptides with non-naturally-occurring amino acids is routine in the art.

In addition, constrained peptides comprising a consensus sequence or a substantially identical consensus sequence variation may be generated by methods well known in the art (Rizo and Gierasch, Ann. Rev. Biochem. 61:387-418 (1992)). For example, constrained peptides may be generated by adding cysteine residues capable of forming disulfide bridges and, thereby, resulting in a cyclic peptide. Cyclic peptides can be constructed to have no free N- or C-termini. Accordingly, they are not susceptible to proteolysis by exopeptidases, although they may be susceptible to endopeptidases, which do not cleave at peptide termini. The amino acid sequences of the peptides with N-terminal or C-terminal D-amino acids and of the cyclic peptides are usually identical to the sequences of the peptides to which they correspond, except for the presence of N-terminal or C-terminal D-amino acid residue, or their circular structure, respectively.

Cyclic Peptides

In some embodiments, a functional equivalent, analogue or derivative of naturally-occurring Ang-(1-7) is a cyclic peptide. As used herein, a cyclic peptide has an intramolecular covalent bond between two non-adjacent residues. The intramolecular bond may be a backbone to backbone, side-chain to backbone or side-chain to side-chain bond (i.e., terminal functional groups of a linear peptide and/or side-chain functional groups of a terminal or interior residue may be linked to achieve cyclization). Typical intramolecular bonds include disulfide, amide and thioether bonds. A variety of means for cyclizing polypeptides are well known in the art, as are many other modifications that can be made to such peptides. For a general discussion, see International Patent Publication Nos. WO 01/53331 and WO 98/02452, the contents of which are incorporated herein by reference. Such cyclic bonds and other modifications can also be applied to the cyclic peptides and derivative compounds of this invention.

Cyclic peptides as described herein may comprise residues of L-amino acids, D-amino acids, or any combination thereof. Amino acids may be from natural or non-natural sources, provided that at least one amino group and at least one carboxyl group are present in the molecule; α- and β-amino acids are generally preferred. Cyclic peptides may also contain one or more rare amino acids (such as 4-hydroxyproline or hydroxylysine), organic acids or amides and/or derivatives of common amino acids, such as amino acids having the C-terminal carboxylate esterified (e.g., benzyl, methyl or ethyl ester) or amidated and/or having modifications of the N-terminal amino group (e.g., acetylation or alkoxycarbonylation), with or without any of a wide variety of side-chain modifications and/or substitutions (e.g., methylation, benzylation, t-butylation, tosylation, alkoxycarbonylation, and the like). Suitable derivatives include amino acids having an N-acetyl group (such that the amino group that represents the N-terminus of the linear peptide prior to cyclization is acetylated) and/or a C-terminal amide group (i.e., the carboxy terminus of the linear peptide prior to cyclization is amidated). Residues other than common amino acids that may be present with a cyclic peptide include, but are not limited to, penicillamine,β,β-tetramethylene cysteine, β,β-pentamethylene cysteine, β-mercaptopropionic acid, β,β-pentamethylene-β-mercaptopropionic acid, 2-mercaptobenzene, 2-mercaptoaniline, 2-mercaptoproline, ornithine, diaminobutyric acid, α-aminoadipic acid, m-aminomethylbenzoic acid and α,β-diaminopropionic acid.

Following synthesis of a linear peptide, with or without N-acetylation and/or C-amidation, cyclization may be achieved by any of a variety of techniques well known in the art. Within one embodiment, a bond may be generated between reactive amino acid side chains. For example, a disulfide bridge may be formed from a linear peptide comprising two thiol-containing residues by oxidizing the peptide using any of a variety of methods. Within one such method, air oxidation of thiols can generate disulfide linkages over a period of several days using either basic or neutral aqueous media. The peptide is used in high dilution to minimize aggregation and intermolecular side reactions. Alternatively, strong oxidizing agents such as I₂ and K₃Fe(CN)₆ can be used to form disulfide linkages. Those of ordinary skill in the art will recognize that care must be taken not to oxidize the sensitive side chains of Met, Tyr, Trp or His. Within further embodiments, cyclization may be achieved by amide bond formation. For example, a peptide bond may be formed between terminal functional groups (i.e., the amino and carboxy termini of a linear peptide prior to cyclization). Within another such embodiment, the linear peptide comprises a D-amino acid. Alternatively, cyclization may be accomplished by linking one terminus and a residue side chain or using two side chains, with or without an N-terminal acetyl group and/or a C-terminal amide. Residues capable of forming a lactam bond include lysine, ornithine (Orn), α-amino adipic acid, m-aminomethylbenzoic acid, α,β-diaminopropionic acid, glutamate or aspartate. Methods for forming amide bonds are generally well known in the art. Within one such method, carbodiimide-mediated lactam formation can be accomplished by reaction of the carboxylic acid with DCC, DIC, ED AC or DCCI, resulting in the formation of an O-acylurea that can be reacted immediately with the free amino group to complete the cyclization. Alternatively, cyclization can be performed using the azide method, in which a reactive azide intermediate is generated from an alkyl ester via a hydrazide. Alternatively, cyclization can be accomplished using activated esters. The presence of electron withdrawing substituents on the alkoxy carbon of esters increases their susceptibility to aminolysis. The high reactivity of esters of p-nitrophenol, N-hydroxy compounds and polyhalogenated phenols has made these “active esters” useful in the synthesis of amide bonds. Within a further embodiment, a thioether linkage may be formed between the side chain of a thiol-containing residue and an appropriately derivatized α-amino acid. By way of example, a lysine side chain can be coupled to bromoacetic acid through the carbodiimide coupling method (DCC, EDAC) and then reacted with the side chain of any of the thiol containing residues mentioned above to form a thioether linkage. In order to form dithioethers, any two thiol containing side-chains can be reacted with dibromoethane and diisopropylamine in DMF.

Exemplary Angiotensin-(1-7) Peptides

In certain aspects, the invention provides linear angiotensin-(1-7) peptides. As discussed above, the structure of naturally-occurring Ang-(1-7) is as follows:

(SEQ ID NO: 1)
Asp¹-Arg²-Val³-Tyr⁴-Ile⁵-His⁶-Pro⁷

The peptides and peptide analogs of the invention can be generally represented by the following sequence:

(SEQ ID NO: 5)
Xaa¹-Xaa²-Xaa³-Xaa⁴-Xaa⁵-Xaa⁶-Xaa⁷,

or a pharmaceutically acceptable salt thereof

Xaa¹ is any amino acid or a dicarboxylic acid. In certain embodiments, Xaa¹ is Asp, Glu, Asn, Acpc (1-aminocyclopentane carboxylic acid), Ala, Me₂Gly (N,N-dimethylglycine), Pro, Bet (betaine, 1-carboxy-N,N,N-trimethylmethanaminium hydroxide), Glu, Gly, Asp, Sar (sarcosine) or Suc (succinic acid). In certain such embodiments, Xaa¹ is a negatively-charged amino acid, such as Asp or Glu, typically Asp.

Xaa² is Arg, Lys, Ala, Cit (citrulline), Orn (ornithine), acetylated Ser, Sar, D-Arg and D-Lys. In certain embodiments, Xaa² is a positively-charged amino acid such as Arg or Lys, typically Arg.

Xaa³ is Val, Ala, Leu, Nle (norleucine), Ile, Gly, Lys, Pro, HydroxyPro (hydroxyproline), Aib (2-aminoisobutyric acid), Acpc or Tyr. In certain embodiments, Xaa³ is an aliphatic amino acid such as Val, Leu, Ile or Nle, typically Val or Nle.

Xaa⁴ is Tyr, Tyr(PO₃), Thr, Ser, homoSer (homoserine), azaTyr (aza-α¹-homo-L-tyrosine) or Ala. In certain embodiments, Xaa⁴ is a hydroxyl-substituted amino acid such as Tyr, Ser or Thr, typically Tyr.

Xaa⁵ is Ile, Ala, Leu, norLeu, Val or Gly. In certain embodiments, Xaa⁵ is an aliphatic amino acid such as Val, Leu, Ile or Nle, typically Ile.

Xaa⁶ is His, Arg or 6-NH₂-Phe (6-aminophenylalaine). In certain embodiments, Xaa⁶ is a fully or partially positively-charged amino acid such as Arg or His.

Xaa⁷ is Cys, Pro or Ala.

In certain embodiments, one or more of Xaa¹-Xaa⁷ is identical to the corresponding amino acid in naturally-occurring Ang-(1-7). In certain such embodiments, all but one or two of Xaa¹-Xaa⁷ are identical to the corresponding amino acid in naturally-occurring Ang-(1-7). In other embodiments, all of Xaa¹-Xaa⁶ are identical to the corresponding amino acid in naturally-occurring Ang-(1-7).

In certain embodiments, Xaa³ is Nle. When Xaa³ is Nle, one or more of Xaa¹-Xaa² and Xaa⁴⁻⁷ are optionally identical to the corresponding amino acid in naturally-occurring Ang-(1-7). In certain such embodiments, all but one or two of Xaa¹-Xaa² and Xaa⁴⁻⁷ are identical to the corresponding amino acid in naturally-occurring Ang-(1-7). In other embodiments, all of Xaa¹-Xaa² and Xaa⁴⁻⁷ are identical to the corresponding amino acid in naturally-occurring Ang-(1-7), resulting in the amino acid sequence: Asp-Arg-Nle-Tyr-Ile-His-Pro (SEQ ID NO:2).

In certain embodiments, the peptide has the amino acid sequence Asp-Arg-Nle-Tyr-Ile-His-Pro (SEQ ID NO:2).

In certain embodiments, the peptide has the amino acid sequence Asp-Arg-Val-Ser-Ile-His-Cys (SEQ ID NO:6) or Asp-Arg-Val-ser-Ile-His-Cys (SEQ ID NO:3).

In some embodiments, a linear angiotensin (1-7) peptide as used herein is a peptide having a sequence of Asp¹-Arg²-Val³-Tyr⁴-Ile⁵-His⁶-Pro⁷-Phe⁸-His⁹ (SEQ ID NO: 23), which is identical to the sequence of Ang(1-9). In some embodiments, an angiotensin (1-7) peptide is a derivative of Ang (1-9). For exemplary Ang (1-9) peptides, including Ang(1-9) derivatives, see U.S. Patent Publication 2012/0172301, the disclosure of which is hereby incorporated by reference.

In some embodiments, a linear angiotensin (1-7) peptide is a peptide with an amino acid sequence of Ala¹-Arg²-Val³-Tyr⁴-Ile⁵-His⁶-Pro⁷(SEQ ID NO: 24). Additional sequences derived from SEQ ID NO: 24 may be found in European Patent Application 2,264,048, the disclosure of which is hereby incorporated by reference.

Further contemplated are variants of the linear peptides described herein, wherein the variants maintain one or more functional properties of the comparator peptide. Variants may have at least 70%, at least 75%, at least 80%, at least 85%, at least 90%, at least 95%, at least 98%, or at least 99% sequence identity to any of the exemplary linear peptides described herein.

Exemplary Cyclic Angiotensin (1-7) Peptides

In certain aspects, the invention provides a cyclic angiotensin-(1-7) (Ang-(1-7)) peptide analog comprising a linkage, such as between the side chains of amino acids corresponding to positions Tyr⁴ and Pro⁷ in Ang. These peptide analogs typically comprise 7 amino acid residues, but can also include a cleavable sequence. As discussed in greater detail below, the invention includes fragments and analogs where one or more amino acids are substituted by another amino acid (including fragments).

Although the following section describes aspects of the invention in terms of a thioether bond linking residues at the 4- and 7-positions, it should be understood that other linkages (as described above) could replace the thioether bridge and that other residues could be cyclized. A thioether bridge is also referred to as a monosulfide bridge or, in the case of Ala-S-Ala, as a lanthionine bridge. Thioether bridge-containing peptides can be formed by two amino acids having one of the following formulas:

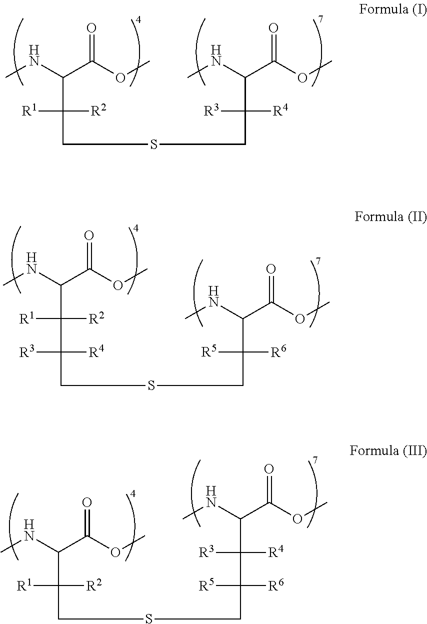

In these formulae, R¹, R², R³, R⁴, R⁵ and R⁶ are independently —H, an alkyl (e.g., C₁-C₆ alkyl, C₁-C₄ alkyl) or an aralkyl group, where the alkyl and aralkyl groups are optionally substituted with one or more halogen, —OH or —NRR′ groups (where R and R′ are independently —H or C₁-C₄ alkyl). In certain embodiments, R¹, R², R³, R⁴, R⁵ and R⁶ are each independently —H or —CH₃, such where all are —H.

In certain embodiments, the invention provides an Ang analog or derivative comprising a thioether bridge according to formula (I). Typically, R¹, R², R³ and R⁴ are independently selected from —H and —CH₃. Peptides comprising a thioether bridge according to formula (I) can be produced, for example, by lantibiotic enzymes or by sulfur extrusion of a disulfide. In one example, the disulfide from which the sulfur is extruded can be formed by D-cysteine in position 4 and L-cysteine in position 7 or by D-cysteine in position 4 and L-penicillamine in position 7 (see, e.g., Galande, Trent and Spatola (2003) Biopolymers 71, 534-551).

In other embodiments, the linkage of the two amino acids can be the bridges depicted in Formula (II) or Formula (III). Peptides comprising a thioether bridge according to Formula (II) can be made, for example, by sulfur extrusion of a disulfide formed by D-homocysteine in position 4 and L-cysteine in position 7. Similarly, peptides comprising a thioether bridge as in Formula (III) can be made, for example, by sulfur extrusion of a disulfide formed by D-cysteine in position 4 and L-homocysteine in position 7.

As discussed above, the Ang analogs and derivatives of the invention vary in length and amino acid composition. The Ang analogs and derivatives of the invention preferably have biological activity or are an inactive precursor molecule that can be proteolytically activated (such as how angiotensin(I), with 10 amino acids, is converted to active fragments by cleavage of 2 amino acids). The size of an Ang analog or derivative can vary but is typically between from about 5 to 10 amino acids, as long as the “core” pentameric segment comprising the 3-7 Nle-thioether-ring structure is encompassed. The amino acid sequence of an analog or derivative of the invention can vary, typically provided that it is biologically active or can become proteolytically activated. Biological activity of an analog or derivative can be determined using methods known in the art, including radioligand binding studies, in vitro cell activation assays and in vivo experiments. See, for example, Godeny and Sayeski, (2006) Am. J. Physiol. Cell. Physiol. 291:C1297-1307; Sarr et al., Cardiovasc. Res. (2006) 71:794-802; and Koziarz et al., (1933) Gen. Pharmacol. 24:705-713.

Ang analogs and derivatives where only the length of the peptide is varied include the following:

a 4,7-cyclized analog designated [Cyc⁴⁻⁷]Ang-(1-7), which is derived from natural Ang-(1-7) (Asp¹-Arg²-Val³-Cyc⁴-Ile⁵-His⁶-Cyc⁷, SEQ ID NO:7).

a 4,7-cyclized analog designated [Nle³, Cyc⁴⁻⁷]Ang-(1-10), which is derived from natural Angiotensin I (Ang-(1-10)) (Asp¹-Arg²-Nle³-Cyc⁴-Ile⁵-His⁶-Cyc⁷-Phe⁸-His⁹-Leu¹⁰, SEQ ID NO:8);

a 4,7-cyclized analog designated [Nle³, Cyc⁴⁻⁷]Ang-(1-8), which is derived from natural Angiotensin II (Ang-(1-8)) (Asp¹-Arg²-Nle³-Cyc⁴-Ile⁵-His⁶-Cyc⁷-Phe⁸, SEQ ID NO:9);

a 4,7-cyclised analog designated [Nle³, Cyc⁴⁻⁷]Ang-(2-8), which is derived from natural Angiotensin III (Ang-(2-8)) (Arg²-Nle³-Cyc⁴-Ile⁵-His⁶-Cyc⁷-Phe⁸, SEQ ID NO:10);

a 4,7-cyclised analog designated [Nle³, Cyc⁴⁻⁷]Ang-(3-8), which is derived from natural Angiotensin IV (Ang-(3-8)) (Nle³-Cyc⁴-Ile⁵-His⁶-Cyc⁷-Phe⁸, SEQ ID NO:11);

a 4,7-cyclised analog designated [Nle³, Cyc⁴⁻⁷]Ang-(1-7) derived from natural Ang-(1-7) (Asp¹-Arg²-Nle³-Cyc⁴-Ile⁵-His⁶-Cyc⁷, SEQ ID NO:12); and

a 4,7-cyclised analog designated [Nle³, Cyc⁴⁻⁷]Ang-(1-9) derived from natural Ang-(1-9) (Asp¹-Arg²-Nle³-Cyc⁴-Ile⁵-His⁶-Cyc⁷-Phe⁸-His⁹, SEQ ID NO:13).

These analogs can have one of the thioether bridges shown in Formulae (I)-(III) as the Cyc⁴⁻⁷ moiety, for example, where Cyc⁴ and Cyc⁷ are represented by Formula (I), such as where R¹-R⁴ are each —H or —CH₃, typically —H.

As compared to the amino acid sequence of the natural angiotensin peptide, the amino acids at positions 4 and 7 of the Cyc⁴⁻⁷ analog are modified to allow introduction of the thioether-ring structures shown above. In addition to the length of the Ang analogs, the amino acids at positions other than 3, 4 and 7 can be the same or different from the naturally-occurring peptide, typically provided that the analog retains a biological function. For analogs of inactive precursors, like [Cyc⁴⁻⁷]Ang-(1-10), biological function refers to one or both of an analog's susceptibility to angiotensin-converting enzymes that can cleave it to a biologically active fragment (e.g. Ang-(1-8) or Ang-(1-7)) or the biological activity of the fragment itself. In certain embodiments, an Ang analog or derivative of the invention has no intrinsic function but inhibits the effects of one or more naturally-occurring angiotensin compounds.

In certain embodiments, an Ang analog of the invention is represented by Formula (IV):

Xaa¹-Xaa²-Xaa³-Cyc⁴-Xaa⁵-Xaa⁶-Cyc⁷ (IV, SEQ ID NO:14)

Xaa¹ is any amino acid, but typically a negatively-charged amino acid such as Glu or Asp, more typically Asp.

Xaa² is a positively-charged amino acid such as Arg or Lys, typically Arg.

Xaa³ is an aliphatic amino acid, such as Leu, Ile or Val, typically Val.

Cyc⁴ forms a thioether bridge in conjunction with Cyc⁷. Cyc⁴ can be a D-stereoisomer and/or a L-stereoisomer, typically a D-stereoisomer. Examples of Cyc⁴ (taken with Cyc⁷) are shown in Formulas (I), (II) and (III). Typically, the R groups in Formulae (I), (II) and (III) are —H or —CH₃, especially —H.

Xaa⁵ is an aliphatic amino acid, such as Leu, Ile or Val, typically Ile.

Xaa⁶ is His.

Cyc⁷ forms a thioether bridge in conjunction with Cyc⁴, such as in Formula (I), (II) or (III). Cyc⁷ can be a D-stereoisomer and/or a L-stereoisomer, typically a L-stereoisomer. Examples of Cyc⁷ (taken with Cyc⁴) are shown in Formulas (I), (II), (III) and (IVIII). Typically, the R groups in FormulaeFormulas (I), (II),) and (III) and (IV) are —H or —CH₃, especially —H.

In certain embodiments, one or more of Xaa¹-Xaa⁶ (excluding Cyc⁴ and Cyc⁷) is identical to the corresponding amino acid in naturally-occurring Ang-(1-7). In certain such embodiments, all but one or two of Xaa¹-Xaa⁶ are identical to the corresponding amino acid in naturally-occurring Ang-(1-7). In other embodiments, all of Xaa¹-Xaa⁶ are identical to the corresponding amino acid in naturally-occurring Ang-(1-7).

In certain embodiments, Cyc⁴ and Cyc⁷ are independently selected from Abu (2-aminobutyric acid) and Ala (alanine), where Ala is present in at least one position. Thus, cyclic analogs can have a thioether linkage formed by -Ala⁴-S-Ala⁷- (Formula (I), where R¹-R⁴ are each —H); -Ala⁴-S-Abu⁷- (Formula (I): R¹-R³ are —H and R⁴ is —CH₃) or -Abu⁴-S-Ala⁷- (Formula (I): R¹, R³ and R⁴ are —H and R² is —CH₃). Specific examples of cyclic analogs comprise a -Abu⁴-S-Ala⁷- or -Ala⁴-S-Ala⁷-linkage.

In certain embodiments, the invention provides an Ang-(1-7) analog with a thioether-bridge between position 4 and position 7 having the amino acid sequence Asp-Arg-Val-Abu-Ile-His-Ala (SEQ ID NO:15) or the amino acid sequence Asp-Arg-Val-Ala-Ile-His-Ala (SEQ ID NO:16), which are represented by the following structural diagrams:

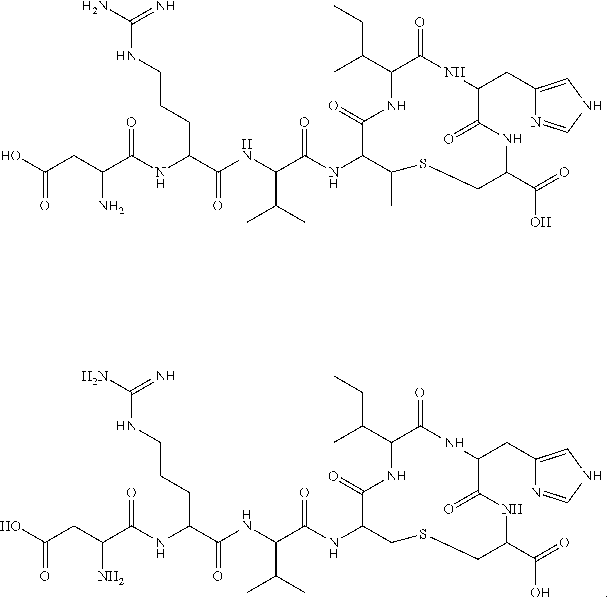 [SMILES: CCC(C)C1NC(=O)C(NC(=O)C(NC(=O)C(CCCNC(=N)N)NC(=O)C(N)CC(=O)O)C(C)C)C(C)SCC(C(=O)O)NC(=O)C(Cc2c[nH]cn2)NC1=O ; CCC(C)C1NC(=O)C(NC(=O)C(NC(=O)C(CCCNC(=N)N)NC(=O)C(N)CC(=O)O)C(C)C)CSCC(C(=O)O)NC(=O)C(Cc2c[nH]cn2)NC1=O]

In certain embodiments, an Ang analog or derivative of the invention is represented by Formula (IV):

Xaa¹-Xaa²-Nle³-Cyc⁴-Xaa⁵-Xaa⁶-Cyc⁷-Xaa⁸-Xaa⁹-Xaa¹⁰ (IV, SEQ ID NO:17)

As discussed above, one or more of Xaa¹, Xaa², Xaa⁸, Xaa⁹ and Xaa¹⁰ are absent in certain embodiments. For example, (1) Xaa¹⁰ is absent, (2) Xaa⁹ and Xaa¹⁰ are absent, (3) Xaa⁸, Xaa⁹ and Xaa¹⁰ are absent, (4) Xaa¹ is absent, (5) Xaa¹ and Xaa¹⁰ are absent, (6) Xaa¹, Xaa⁹ and Xaa¹⁰ are absent, (7) Xaa¹, Xaa⁸, Xaa⁹ and Xaa¹⁰ are absent, (8) Xaa¹ and Xaa² are absent, (9) Xaa¹, Xaa² and Xaa¹⁰ are absent, (10) Xaa¹, Xaa², Xaa⁹ and Xaa¹⁰ are absent, or (11) Xaa¹, Xaa², Xaa⁸, Xaa⁹ and Xaa¹⁰ are absent. For each of these embodiments, the remaining amino acids have the values described below.

Xaa¹, when present, is any amino acid, but typically a negatively charged amino acid such as Glu or Asp, more typically Asp.

Xaa², when present, is a positively charged amino acid such as Arg or Lys, typically Arg.

Nle³ is norleucine.

Cyc⁴ forms a thioether bridge in conjunction with Cyc⁷. Cyc⁴ can be a D-stereoisomer and/or a L-stereoisomer, typically a D-stereoisomer. Examples of Cyc⁴ (taken with Cyc⁷) are shown in Formulas (I), (II) and (III). Typically, the R groups in Formulae (I), (II) and (III) are —H or —CH₃, especially —H.

Xaa⁵ is an aliphatic amino acid, such as Leu, Nle, Ile or Val, typically Ile.

Xaa⁶ is His.

Cyc⁷ forms a thioether bridge in conjunction with Cyc⁴, such as in Formula (I), (II) or (III). Cyc⁷ can be a D-stereoisomer and/or a L-stereoisomer, typically a L-stereoisomer. Examples of Cyc⁷ (taken with Cyc⁴) are shown in Formulas (I), (II) and (III). Typically, the R groups in Formulae (I), (II) and (III) are —H or —CH₃, especially —H.

Xaa⁸, when present, is an amino acid other than Pro, typically Phe or Ile. In certain embodiments, Ile results in an inhibitor of Ang(1-8). In certain embodiments, Phe maintains the biological activity of Ang(1-8) or Ang(1-10).

Xaa⁹, when present, is His.

Xaa¹⁰, when present, is an aliphatic residue, for example, Ile, Val or Leu, typically Leu.

In certain embodiments, one or more of Xaa¹-Xaa¹⁰ (excluding Nle³, Cyc⁴ and Cyc⁷) is identical to the corresponding amino acid in naturally-occurring Ang (including Ang-(1-7), Ang(1-8), Ang(1-9), Ang(1-10), Ang(2-7), Ang(2-8), Ang(2-9), Ang(2-10), Ang(3-8), Ang(3-9) and Ang(3-10). In certain such embodiments, all but one or two of Xaa¹-Xaa¹⁰ (for those present) are identical to the corresponding amino acid in naturally-occurring Ang. In other embodiments, all of Xaa¹-Xaa¹⁰ (for those present) are identical to the corresponding amino acid in naturally-occurring Ang.

In certain embodiments, Cyc⁴ and Cyc⁷ are independently selected from Abu (2-aminobutyric acid) and Ala (alanine), where Ala is present at at least one position. Thus, encompassed are cyclic analogs comprising a thioether linkage formed by -Ala⁴-S-Ala⁷-(Formula (I), where R¹-R⁴ are each —H); -Ala⁴-S-Abu⁷- (Formula (I): R¹-R³ are —H and R⁴ is —CH₃) or -Abu⁴-S-Ala⁷- (Formula (I): R¹, R³ and R⁴ are —H and R² is —CH₃). Specific cyclic analogs comprise a -Abu⁴-S-Ala⁷- or -Ala⁴-S-Ala⁷-linkage.

In particular, the invention provides an Ang-(1-7) analog or derivative with a thioether-bridge between position 4 and position 7 having the amino acid sequence Asp-Arg-Nle-Abu-Ile-His-Ala (SEQ ID NO:18) or the amino acid sequence Asp-Arg-Nle-Ala-Ile-His-Ala (SEQ ID NO:19).

In another aspect, the invention provides an Ang-(1-8) analog or derivative with a thioether-bridge between position 4 and position 7 having Ang-(1-8) antagonistic activity, in particular an Ang(1-8) analog or derivative having the amino acid sequence Asp-Arg-Nle-Abu-Ile-His-Ala-Ile (SEQ ID NO:20), the amino acid sequence Asp-Arg-Nle-Ala-Ile-His-Ala-Ile (SEQ ID NO:21) or the amino acid sequence Asp-Arg-Nle-Abu-Ile-His-Ala-Ile (SEQ ID NO:22).

An alkyl group is a straight chained or branched non-aromatic hydrocarbon that is completely saturated. Typically, a straight chained or branched alkyl group has from 1 to about 20 carbon atoms, preferably from 1 to about 10. Examples of straight chained and branched alkyl groups include methyl, ethyl, n-propyl, iso-propyl, n-butyl, sec-butyl, tert-butyl, pentyl, hexyl, pentyl and octyl. A C1-C4 straight chained or branched alkyl group is also referred to as a “lower alkyl” group.

An aralkyl group is an alkyl group substituted by an aryl group. Aromatic (aryl) groups include carbocyclic aromatic groups such as phenyl, naphthyl, and anthracyl, and heteroaryl groups such as imidazolyl, thienyl, furyl, pyridyl, pyrimidyl, pyranyl, pyrazolyl, pyrrolyl, pyrazinyl, thiazolyl, oxazolyl, and tetrazolyl. Aromatic groups also include fused polycyclic aromatic ring systems in which a carbocyclic aromatic ring or heteroaryl ring is fused to one or more other heteroaryl rings. Examples include benzothienyl, benzofuryl, indolyl, quinolinyl, benzothiazole, benzoxazole, benzimidazole, quinolinyl, isoquinolinyl and isoindolyl.

Angiotensin (1-7) peptides, including derivatives and analogs, may be present in varying amounts in various embodiments. For example, an angiotensin (1-7) peptide is present in an amount ranging from about 10-1000 mg (e.g., about 20 mg-1,000 mg, 30 mg-1,000 mg, 40 mg-1,000 mg, 50 mg-1,000 mg, 60 mg-1,000 mg, 70 mg-1,000 mg, 80 mg-1,000 mg, 90 mg-1,000 mg, about 10-900 mg, 10-800 mg, 10-700 mg, 10-600 mg, 10-500 mg, 100-1000 mg, 100-900 mg, 100-800 mg, 100-700 mg, 100-600 mg, 100-500 mg, 100-400 mg, 100-300 mg, 200-1000 mg, 200-900 mg, 200-800 mg, 200-700 mg, 200-600 mg, 200-500 mg, 200-400 mg, 300-1000 mg, 300-900 mg, 300-800 mg, 300-700 mg, 300-600 mg, 300-500 mg, 400 mg-1,000 mg, 500 mg-1,000 mg, 100 mg-900 mg, 200 mg-800 mg, 300 mg-700 mg, 400 mg-700 mg, and 500 mg-600 mg). In some embodiments, an angiotensin (1-7) peptide is present in an amount of or greater than about 10 mg, 50 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg. In some embodiments, an angiotensin (1-7) peptide is present in an amount of or less than about 1000 mg, 950 mg, 900 mg, 850 mg, 800 mg, 750 mg, 700 mg, 650 mg, 600 mg, 550 mg, 500 mg, 450 mg, 400 mg, 350 mg, 300 mg, 250 mg, 200 mg, 150 mg, or 100 mg.

In some embodiments, a cyclic angiotensin (1-7) peptide is a cyclized Ang (1-9) peptide or a cyclized peptide comprising SEQ ID NO: 24.

Further contemplated are variants of the cyclic peptides described herein, wherein the variants maintain one or more functional properties of the comparator peptide. Cyclized variants may have a sequence at least 70%, at least 75%, at least 80%, at least 85%, at least 90%, at least 95%, at least 98%, or at least 99% sequence identity to any of the sequences of the exemplary cyclic peptides described herein.

pH-Lowering Agents

It is contemplated that a pH-lowering agent suitable for the present invention include any pharmaceutically acceptable pH-lowering agent, or combination of pH-lowering agents, that are a) not toxic to the gastrointestinal tract, b) are capable of either delivering hydrogen ions or capable of inducing higher hydrogen ion content from the local environment, and/or c) that are capable of being orally administered in an amount sufficient to lower the local intestinal pH below the pH optima for proteases found there. Various tests may be used to determine if a pH-lowering agent is suitable for the present invention and what amount is appropriate. For example, a pH-lowering agent or combination of pH-lowering agents is suitable for the present invention if, a particular amount when added to a solution of 10 milliliters of 0.1M sodium bicarbonate lowers the pH of the solution to no higher than 5.5, 4.7, or 3.5. In some embodiments, an amount of pH-lowering agent or agents may be added to lower pH, in a solution of 10 milliliters of 0.1M sodium bicarbonate, to no higher than 3.4, 3.2, 3.0, or 2.8.

In some embodiments, a suitable pH-lowering agent or agents include at least one pH-lowering agent that has a pKa no higher than 4.2 (e.g., no higher than 4.0, 3.8, 3.6, 3.4, 3.2, 3.0 or 2.8). Exemplary pH-lowering agents suitable for the present invention include, but are not limited to, carboxylic acids such as acetylsalicylic, acetic, ascorbic, citric, fumaric, glucuronic, glutaric, glyceric, glycocolic, glyoxylic, isocitric, isovaleric, lactic, maleic, oxaloacetic, oxalosuccinic, propionic, pyruvic, succinic, tartaric, and valeric; aluminum chloride; zinc chloride; acid salts of amino acids (or derivatives thereof) including acid salts of acetylglutamic acid, alanine, arginine, asparagine, aspartic acid, betaine, carnitine, carnosine, citrulline, creatine, glutamic acid, glycine, histidine, hydroxylysine, hydroxyproline, hypotaurine, isoleucine, leucine, lysine, methylhistidine, norleucine, ornithine, phenylalanine, proline, sarcosine, serine, taurine, threonine, tryptophan, tyrosine, and valine; certain phosphate esters including fructose 1,6 diphosphate and glucose 1,6 diphosphate may also be appropriate pH-lowering agents in certain embodiments. In particular embodiments, citric acid or tartaric acid is used as pH-lowering agent.

The quantity required of any particular pH-lowering agent or combination of pH-lowering agents may vary. Typically, suitable amount may be determined using various tests known in the art and described herein (for example, using pH-lowering test in a solution of 10 milliliters of 0.1M sodium bicarbonate described above). As non-limiting examples, suitable amount of a pH lowering agent used in a formulation according to the present invention may be an amount of or greater than about 100 mg, 200 mg, 250 mg, 300 mg, 350 mg, 400 mg, 425 mg, 450 mg, 475 mg, 500 mg, 525 mg, 550 mg, 575 mg, 600 mg, 625 mg, 650 mg, 675, mg, 700 mg, 725 mg, 750 mg, 775 mg, 800 mg, 825 mg, 850 mg, 875 mg, 900 mg, 925 mg, 950 mg, 975 mg, or 1,000 mg. In other embodiments, the amount of citric acid used may exceed 1,000 mg.

In some embodiments, a suitable amount of a pH lowering agent (e.g., citric acid or tartaric acid) used may be measured as a percent of the total weight of a particular dosage form. As non-limiting examples, a suitable amount of a pH lowering agent used may be an amount of or greater than about 10% (e.g., of or greater than 15%, 20%, 25%, 30%, 35%, 40%, 45%, 50%, 55%, 60%, 65%, 70%, 75%, 80%, 85%, 90%, or 95%) of the total weight of a solid dosage form.

### Absorption Enhancers

In various embodiments, a formulation of the invention has one or more absorption enhancers. As used herein, an absorption enhancer refers to an agent that increase the solubility of other components in either the aqueous or lipophilic environment into which they are released and/or enhance the uptake of an active peptide (e.g., an angiotensin (1-7) peptide) across the intestinal wall. In some embodiments, an absorption enhancer is referred to as a solubility enhancer and/or an uptake enhancer.

In some embodiments, it is possible to have a mixture of absorption enhancers wherein some provide enhanced solubility, some provide enhanced uptake, and some provide both. It is possible to have various numbers of absorption enhancers in a given embodiment including, without limitation, one, two, three, four, five, six, seven, eight, nine, or ten absorption enhancers.

Surface active agents are an example of useful absorption enhancers with properties of both solubility enhancers and uptake enhancers. In some embodiment, when surface active agents are used as absorption enhancers, they may be free flowing powders for facilitating the mixing and loading of capsules during the manufacturing process. In other embodiments when a surface active agent is used to increase the bioavailability of an angiotensin (1-7) peptide, the surface active agent may be selected from the group consisting of (a) anionic surface active agents such as cholesterol derivatives (e.g. bile acids), (b) cationic surface agents (e.g. acyl carnitines, phospholipids and the like), (c) non-ionic surface active agents, and (d) mixtures of anionic surface active agents and negative charge neutralizers, and combinations thereof. Negative charge neutralizers include but are not limited to acyl carnitines, cetyl pyridinum chloride, and the like.

In some embodiments, an acid soluble bile acid and a cationic surface active agent with be used together as absorption enhancers. Acyl carnitines (such as lauroyl carnitine), phospholipids and bile acids may be particularly effective absorption enhancers in some embodiments.

While a variety of absorption enhancers are suitable for use in various embodiments, the following exemplary list is intended to illustrate some embodiments of the invention. Without limitation, some suitable absorption enhancers include: (a) salicylates such as sodium salicylate, 3-methoxysalicylate, 5-methoxysalicylate and homovanilate; (b) bile acids such as taurocholic, tauorodeoxycholic, deoxycholic, cholic, glycholic, lithocholate, chenodeoxycholic, ursodeoxycholic, ursocholic, dehydrocholic, fusidic, etc.; (c) non-ionic surfactants such as polyoxyethylene ethers (e.g. Brij 36T, Brij 52, Brij 56, Brij 76, Brij 96, Texaphor A6, Texaphor A14, Texaphor A60 etc.), p-t-octyl phenol polyoxyethylenes (Triton X-45, Triton X-100, Triton X-114, Triton X-305 etc.) nonylphenoxypoloxyethylenes (e.g. Igepal CO series), polyoxyethylene sorbitan esters (e.g. Tween-20, Tween-80 etc.); (d) anionic surfactants such as dioctyl sodium sulfosuccinate; (e) lyso-phospholipids such as lysolecithin and lysophosphatidylethanolamine; (f) acylcarnitines, acylcholines and acyl amino acids such as lauroylcarnitine, myristoylcarnitine, palmitoylcarnitine, lauroylcholine, myristoylcholine, palmitoylcholine, hexadecyllysine, N-acylphenylalanine, N-acylglycine etc.; g) water soluble phospholipids such as diheptanoylphosphatidylcholine, dioctylphosphatidylcholine etc.; (h) medium-chain glycerides which are mixtures of mono-, di- and triglycerides containing medium-chain-length fatty acids (caprylic, capric and lauric acids); (i) ethylene-diaminetetraacetic acid; (j) cationic surfactants such as cetylpyridinium chloride; (k) fatty acid derivatives of polyethylene glycol such as Labrasol, Labrafac, etc.; and (l) alkylsaccharides such as lauroyl maltoside, lauroyl sucrose, myristoyl sucrose, palmitoyl sucrose, etc.

In some embodiments, the absorption enhancer(s) will be present in a quantity measured as a percent by weight, relative to the overall weight of the pharmaceutical composition (typically exclusive of enteric coating). By way of additional non-limiting example, the quantity of absorption enhancer present in an embodiment may range from 0.1 to 20 percent by weight; from 0.5 to 20 percent by weight; from 1.0 to 20 percent by weight, from 2.0 to 20 percent by weight, from 3.0 to 20 percent by weight, from 4.0 to 20 percent by weight, from from 5.0 to 20 percent by weight, from 5.0 to 15 percent by weight, from 5.0 to 14 percent by weight, from 5.0 to 13 percent by weight, from 5.0 to 12 percent by weight, from 5.0 to 12 percent by weight, from 5.0 to 11 percent by weight, from 5.0 to 10 percent by weight, from 6.0 to 10 percent by weight, from 7.0 to 10 percent by weight, from 8.0 to 10 percent by weight, from 9.0 to 10 percent by weight, from 5.0 to 9.0 percent by weight, from 5.0 to 8.0 percent by weight, from 5.0 to 7.0 percent by weight, and from 5.0 to 6.0 percent by weight.

In some embodiments, the weight ratio of pH-lowering agent(s) to absorption enhancer(s) may be about 3:1, 4:1, 5:1, 6:1, 7:1, 8:1, 9:1, 10:1, 11:1, 12:1, 13:1, 14:1, 15:1, 16:1, 17:1, 18:1, 19:1, 20:1 or between any two of the foregoing exemplary ratios. The total weight of all pH-lowering agents and the total weight of all absorption enhancers in a given pharmaceutical composition is included in the foregoing exemplary ratios. For example, if a pharmaceutical composition includes two pH-lowering agents and three absorption enhancers, the foregoing ratios will be computed on the total combined weight of both pH-lowering agents and the total combined weight of all three absorption enhancers.

In some embodiments, the absorption enhancer(s) will be soluble at acid pH, such as less than pH 5.5, and in particular, between pH 3.0 and pH 5.0.

### Protective Vehicles

As used herein, a protective vehicle refers to any protective component and/or structure, such as a carrier, a layer, a coating or other vehicle, that protects an active peptide (e.g., an angiotensin (1-7) peptide) from stomach proteases. Typically, a protective vehicle dissolves eventually so that the active and other ingredients in a particular dosage form may be released. A common form of protective vehicle is an enteric coating. In some embodiments, a suitable enteric costing may prevent breakdown of the pharmaceutical composition of the invention in 0.1N HCl for at least two hours, then capable of permitting complete release of all contents of the pharmaceutical composition within thirty minutes after pH is increased to 6.3 in a dissolution bath in which said composition is rotating at 100 revolutions per minute.

Many enteric coatings are known in the art and are useful in one or more embodiments. Non-limiting examples of enteric coatings include cellulose acetate phthalate, hydroxypropyl methylethylcellulose succinate, hydroxypropyl methylcellulose phthalate, carboxyl methylethylcellulose and methacrylic acid-methyl methacrylate copolymer. In some embodiments, an angiotensin (1-7) peptide, absorption enhancers such as solubility and/or uptake enhancer(s), and pH-lowering agent(s), are included in a sufficiently viscous protective syrup to permit protected passage of the components of the embodiment through the stomach.

Suitable enteric coatings may be applied, for example, to capsules after the active and other components of the invention have been loaded within the capsule. In other embodiments, enteric coating is coated on the outside of a tablet or coated on the outer surface of particles of active components which are then pressed into tablet form, or loaded into a capsule.

In some embodiments it may be desirable that all components of the invention be released from the carrier or vehicle, and solubilized in the intestinal environment as simultaneously as possible. It may also be preferred in some embodiments that the vehicle or carrier release the active components in the small intestine where uptake enhancers that increase transcellular or paracellular transport are less likely to cause undesirable side effects than if the same uptake enhancers were later released in the colon. It will be appreciated, however, that the present invention is believed effective in the colon as well as in the small intestine. Numerous vehicles or carriers, in addition to the ones discussed above, are known in the art.

In some embodiments, it may be desirable (especially in optimizing how simultaneously the components of the invention are released) to keep the amount of enteric coating low. In some embodiments, an enteric coating adds no more than 30% to the weight of the remainder of pharmaceutical composition such as a solid dosage form (the “remainder” being the pharmaceutical composition exclusive of enteric coating itself). In other embodiments, an enteric coating adds less than 20%, less than 19%, less than 18%, less than 17%, less than 16%, less than 15%, less than 14%, less than 13%, less than 12%, less than 11%, or less than 10%. In some embodiments, a protective vehicle such as an enteric coating constitutes an amount of or less than approximately 25%, 24%, 23%, 22%, 21%, 20%, 19%, 18%, 17%, 16%, 15%, 14%, 13%, 12%, 11%, 10%, 9%, 8%, 7%, 6%, 5% of the total weight of a pharmaceutical composition (e.g., a solid dosage form).

### Dosage Forms

As used herein, dosage forms refer to a mixture of active drug components and nondrug components. Various dosage forms may be used according to the invention, including but not limited to, liquid dosage forms, solid dosage forms and semisolid dosage forms. Common dosage forms include pill, tablet, capsule, drink or syrup. In some embodiments, solid dosage forms such as pill, tablet or capsule are used.

Typically, a particularly desirable dosage form provides simultaneous release of the angiotensin-(1-7) peptide, pH-lowering agent and absorption enhancers. This is highly desirable because the acid is best able to reduce undesirable proteolytic attack on the peptide when the acid is released in close time proximity to release of the peptide. Near simultaneous release is best achieved by administering all components of the invention as a single pill, tablet or capsule.

Various embodiments may optionally include common pharmaceutical excipients such as diluents, glycants, lubricants, gelatin capsules, preservatives, colorants and the like in their usual known sizes and amounts. Exemplary, non-limiting excipients include pro-salts, polyplastum and sodium stearyl fumerate. In some embodiments, another peptide (such as albumin, casein, soy protein, other animal or vegetable proteins and the like) is included to reduce non-specific adsorption (e.g., binding of angiotensin (1-7) peptide to the intestinal mucus barrier) thereby lowering the necessary concentration of the expensive peptide active agent. When added, the additional peptide is typically from 1.0 to 10.0 percent by weight relative to the weight of the overall pharmaceutical composition (excluding protective vehicle). Typically, this additional peptide is not physiologically active and is most preferably a food peptide such as soy bean peptide or the like. Without intending to be bound by theory, this second peptide may also increase bioavailability by acting as a protease scavenger that desirably competes with the peptide active agent for protease interaction. The second peptide may also aid the active compound's passage through the liver.

In some embodiments, the pH-lowering agent(s), the angiotensin (1-7) peptide, the absorption enhancer(s) and other excipients (whether single compounds or a plurality of compounds in each category) be uniformly dispersed in a dosage form. In other embodiments, a dosage form may comprise granules that include a pharmaceutical binder having the angiotensin (1-7) peptide, the pH-lowering agent and the absorption enhancer uniformly dispersed within said binder. In yet other embodiments, granules may also consist of an acid core, surrounded by a uniform layer of organic acid, a layer of enhancer and a layer of peptide that is surrounded by an outer layer of organic acid. Granules may be prepared from an aqueous mixture consisting of pharmaceutical binders such as polyvinyl pyrrolidone or hydroxypropyl methylcellulose, together with the pH-lowering agents, absorption enhancers and peptide active agents of the invention.

As described, various embodiments may have differing amounts of ingredients and differing ingredients as well. Regardless of the recipe of a particular embodiment, the total weight of all ingredients present in that embodiment may fall within a certain weight range, such as from about 500-1500 (e.g., from about 500-1200 mg, 500-1000 mg, 600-1500 mg, 600-1200 mg, 600-1000 mg, 700-1500 mg, 700-1200 mg, 700-1000 mg, 800-1500 mg, 800-1200 mg, 800-1000 mg). In some embodiments, a suitable solid dosage form has a total weight of or greater than about 500 mg, 600 mg, 700 mg, 800 mg, 900 mg, 1000 mg, 1100 mg, 1200 mg, 1300 mg, 1400 mg, or 1500 mg. In some embodiments, a suitable solid dosage form has a total weight of or less than about 2000 mg, 1900 mg, 1800 mg, 1700 mg, 1600 mg, 1500 mg, 1400 mg, 1300 mg, 1200 mg, 1100 mg, 1000 mg, 900 mg, 800 mg, 700 mg, 600 mg, or 500 mg.

### EXAMPLES

### Example 1

### Oral Delivery of Angiotensin (1-7) Peptides

This example demonstrates that angiotensin (1-7) can be effectively delivered orally using an exemplary formulation according to the present invention. Specifically, the feasibility of orally delivering an angiotensin (1-7) peptide was demonstrated by administrating it in a liquid formulation to an anesthetized rat via intra-duodenal injection (ID). This model mimics the release and absorption expected from an orally delivered enteric coated solid dosage form such as capsule or tablet.

Initially, a baseline pharmacokinetic profile in female Sprague-Dawley rats was obtained by subcutaneous (SC) administration of angiotensin (1-7) in phosphate buffered saline (PBS). Blood samples (0.6 ml) was taken from a cannula implanted into the right carotid artery before and 5, 10, 20, 30, 60 and 90 minutes after the injection of the peptide and replaced with an equal volume of heparinized saline.

After extraction, the samples were then transferred to ice-cold tubes containing a protease inhibitor cocktail. The samples were kept on ice until they were centrifuged at 4° C. to obtain plasma. The plasma supernatants were then frozen −70° C. until analysis using an LC-MS assay.

Table 1 summarized exemplary individual baseline A(1-7) levels achieved at the designated time points and the non-compartmental PK values. Three rats were administered 0.3 mL of 10 mg/mL A(1-7) as a subcutaneous injection. The pharmacokinetics of A(1-7) were determined using a non-compartmental model, where individual pharmacokinetics were determined. The mean concentration for each time point was calculated and the PK values for these mean values were estimated. The Tmax was achieved approximately 10 to 90 minutes after administration. Half-lives are approximately 15 minutes for this treatment group. Total mean A(1-7) exposure over the observation period was 614 ng*min/mL with a range of 585 to 656 ng*min/mL.

TABLE 1

| | 0.3 mL: A(1-7) Subcutaneous injection | | | | |
|---|---|---|---|---|---|
| | Angiotensin (1-7) (ng/mL) | | | | |
| | | | | Mean PK | |
| Time Point (min) | Rat1 | Rat 2 | Rat 3 | Value | Average |
| 0 | 0.121 | 10.2 | 1.41 | | 4.50 |
| 5 | 0.120 | 7.47 | 4.37 | | 3.99 |
| 10 | 18.6 | 0.94 | 1.09 | | 6.88 |
| 20 | 6.25 | 15.7 | 5.71 | | 9.22 |
| 40 | 15.30 | 6.38 | 4.47 | | 8.72 |
| 60 | 1.03 | 2.65 | 7.46 | | 3.71 |
| 90 | 1.14 | 6.59 | 17.4 | | 8.38 |
| Cmax (ng/mL) | 18.6 | 15.7 | 17.4 | 17.2 | 9.2 |
| Tmax (ng/mL) | 10 | 20 | 90 | 40.0 | 20 |
| Half-Life (min) | 15 | 15.58 | | 15.1 | |
| AUC₀₋₉₀ (ng*min/mL) | 583 | 598.1 | 656.1 | 612.4 | 614 |

The oral delivery of an angiotensin (1-7) peptide (e.g., TXA127) was then evaluated in a rat model that mimics the release of the peptide into the intestine by enteric-coated capsule. Briefly, the duodenums of anesthetized rats were surgically exposed, and an angiotensin (1-7) peptide was delivered through a 27 gauge needle into the duodenum. A baseline was obtained by ID administration of an angiotensin (1-7) in PBS. Samples of blood was removed from the carotid artery before and 5, 10, 20, 40, 60 and 90 minutes after peptide administration. Subsequently, an angiotensin (1-7) peptide was administered ID in 400 mM citrate buffer (pH 3.5) and lauroyl-L-carnitine (LLC) (10 mg/ml), a formulation that mimics the contents of the enteric-coated capsules. In order to maximize the stability of TXA127 in rat circulation, captopril (e.g., 0.5 mg/ml or 5 mg/ml) may be added to the formulations. Blood samples were taken at the same time points as the baseline study and handled as described above for analysis. Exemplary results were summarized in Tables 2-5.

Table 2 summarizes exemplary results from a total of 6 rats treated with 0.3 mL of 10 mg/mL Angiotensin (1-7) (A(1-7)) formulated in PBS. The pharmacokinetics of A(1-7) were determined using a non-compartmental model. The average concentration for each time point was calculated and the PK values for these mean values were estimated. These were compared with the mean PK values, which were calculated by taking the average of all individual PK parameters. The Tmax was achieved approximately 10 to 60 minutes after administration. Half-lives ranged from 7 to 140 minutes for this treatment group. Total mean A(1-7) exposure over the observation period was 403 ng*min/mL with a range of 123 to 881 ng*min/mL.

TABLE 2

| | 0.3 ml A, ID [10 mg/ml Angiotensin (1-7) in PBS] | | | | | | | |
|---|---|---|---|---|---|---|---|---|
| | Angiotensin (1-7) (ng/mL) | | | | | | | |
| | | | | | | | Mean | |
| Time Point | | | | | | | PK | |
| (min) | Rat 1 | Rat 2 | Rat 1 | Rat 2 | Rat 1 | Rat 2 | Values | Average |
| 0 | 0.23 | 0.0 | 1.54 | 1.00 | 1.84 | 5.81 | | 1.74 |
| 5 | 0.84 | 12.3 | 0.94 | 0.88 | 0.891 | 9.24 | | 4.18 |
| 10 | 3.44 | 6.38 | 1.08 | 0.99 | 1.12 | 4.08 | | 2.85 |
| 20 | 0.62 | 13.3 | 1.91 | 1.79 | 1.80 | 2.05 | | 3.58 |
| 40 | 0.27 | 15.4 | 1.22 | 4.14 | 1.30 | 3.89 | | 4.37 |
| 60 | 1.55 | 6.27 | 3.97 | 8.75 | 3.07 | 10.4 | | 5.67 |
| 90 | 2.61 | 7.19 | 0.78 | 7.54 | 1.23 | 10.4 | | 4.96 |
| Cmax (ng/mL) | 3.44 | 15 | 4 | 9 | 3 | 10.4 | 7.5 | 5.7 |
| Tmax (ng/mL) | 10 | 40 | 60 | 60 | 60 | | 46.0 | 60 |
| Half-Life (min) | | 51 | 13 | 140 | 23 | 7 | 38.9 | 155 |
| AUC₀₋₉₀ | 123 | 881 | 181 | 456 | 166 | 616 | 403 | 404 |
| (ng*min/mL) | | | | | | | | |

Table 3 summarizes exemplary A(1-7) concentration found in rats treated with 0.3 mL of 10 mg/mL A(1-7) in preparation containing 10 mg/mL LLC, 400 mM Citrate, 150 mM NaCl pH 3.5. The pharmacokinetics of A(1-7) was determined using a non-compartmental model. Additionally, the mean concentration for each time point was calculated and the PK values for these mean values were estimated. These mean concentration values were compared with the mean PK values, which is calculated by taking the average of all individual PK parameters. The Tmax was achieved approximately 5 to 10 minutes after administration. Half-lives ranged from 9.3 to 173.3 minutes for this treatment group. Total mean A(1-7) exposure over the observation period was 4,274 ng*min/mL with a range of 422 to 19,502 ng*min/mL.

TABLE 3

| | 0.3 ml B, ID [10 mg/ml Angiotensin (1-7), 10 mg/ml LLC, | | | | | | | |
|---|---|---|---|---|---|---|---|---|
| | 400 mM Citrate pH 3.5, 150 mM NaCl] | | | | | | | |
| | Angiotensin (1-7) (ng/mL) | | | | | | | |
| | | | | | | | Mean | |
| Time Point | | | | | | | PK | |
| (min) | Rat 3** | Rat 4 | Rat 3 | Rat 4 | Rat 3 | Rat 4 | Values | Average |
| 0 | 1.46 | 0.171 | 3.02 | 1.31 | 1.81 | 1.29 | | 1.51 |
| 5 | >1000 | 259 | 3.48 | 89.7 | 60.7 | 56.38 | | 416.54 |
| 10 | >1000 | 21 | 19.3 | 15.2 | 59.6 | 17.92 | | 203.84 |
| 20 | 59.9 | 7.35 | 6.02 | 18.4 | 51.2 | 6.02 | | 24.82 |
| 40 | 4.88 | 5.6 | 2.32 | 5.05 | 11.7 | 1.11 | | 5.11 |
| 60 | 4.74 | 1.7 | 3.99 | 6.36 | 10.9 | 3.48 | | 5.20 |
| 90 | 3.93 | 3.99 | 1.11 | 4.43 | 4.74 | 3.39 | | 3.60 |
| Cmax (ng/mL) | >1000 | 259.0 | 19.3 | 89.7 | 60.7 | 56.4 | 419.2 | 416.5 |
| Tmax (ng/mL) | 5.0 | 5.0 | 10.0 | 5.0 | 5.0 | 5.0 | 5.8 | 5 |
| Half-Life (min) | 9.3 | 173.3 | 23.8 | 25.2 | 21.6 | 25.0 | 46.4 | 13 |
| AUC₀₋₉₀ | 19,502.2 | 1,777.5 | 422.8 | 1,168.2 | 2,100.6 | 669.9 | 4,273.5 | 4274 |
| (ng*min/mL) | | | | | | | | |

**data for this rat was not included in averages or analysis

Table 4 summarizes exemplary pharmacokinetics results from seven rats administered with 0.3 mL of 10 mg/mL A(1-7) in a preparation containing 0.5 mg/mL captopril, 10 mg/mL LLC, 400 mM Citrate, 150 mM NaCl at pH 3.5. The pharmacokinetics of A(1-7) were determined using a non-compartmental model. The average concentration for each time point was calculated and the PK values for these mean values were estimated. These mean concentration values were compared with the mean PK values, which is calculated by taking the average of all individual PK parameters. AUCs were determined using values >1,000 ng/mL to provide a perspective of the range of exposure. In addition mean concentration for each time point was calculated and the PK values for these mean values were estimated. The Tmax was achieved approximately 5 to 10 minutes after administration. Half-lives ranged from 11.9 to 29.1 minutes for this treatment group. Total mean A(1-7) exposure over the observation period was 7,152 ng*min/mL with a range of 1,969 to 9,257 ng*min/mL.

TABLE 4

| | 0.3 ml C, ID [10 mg/ml Angiotensin (1-7), Captopril 0.5 mg/ml, | | | | | | | |
|---|---|---|---|---|---|---|---|---|
| | 10 mg/ml LLC, 400 mM Citrate pH 3.5, 150 mM NaCl] | | | | | | | |
| | Angiotensin (1-7) (ng/mL) | | | | | | | |
| | | | | | | | Mean | |
| Time Point | | | | | | | PK | |
| (min) | Rat 5 | Rat 6** | Rat 5 | Rat 6 | Rat 5 | Rat 6 | Values | Average |
| 0 | 1.24 | 1.13 | 1.54 | 0.51 | 1.29 | 0.98 | | 1.1 |
| 5 | 879.00 | >1000 | 468.00 | 57.20 | 735.00 | 325.00 | | 628.31 |
| 10 | 407.00 | 349.00 | 313.00 | 83.00 | 248.00 | 145.67 | | 233.21 |
| 20 | 88.70 | 496.00 | 141.00 | 64.10 | 16.30 | 70.14 | | 129.13 |
| 40 | 4.24 | 19.60 | 17.30 | 7.68 | 8.42 | 22.85 | | 13.87 |
| 60 | 12.30 | 10.10 | 4.96 | 6.16 | 12.30 | 37.30 | | 14.45 |
| 90 | 5.61 | 5.83 | 7.84 | 4.93 | 15.80 | 7.36 | | 7.74 |
| Cmax (ng/mL) | 879.0 | >1000 | 468.0 | 83.0 | 735.0 | 83.0 | 609.3 | 628.3 |
| Tmax (ng/mL) | 5.0 | 5.0 | 5.0 | 10.0 | 5.0 | 10.0 | 7.1 | 5 |
| Half-Life (min) | 11.9 | 29.1 | 13.9 | 18.3 | 17.9 | 18.3 | 19.4 | 14 |
| AUC₀₋₉₀ | | | | | | | | |
| (ng*min/mL) | 9,257 | 20,442 | 7,394 | 2,252 | 6,495 | 2,252 | 7,152 | 7,587 |

**data for this rat was not included in averages or analysis

Table 5 summarizes exemplary A(1-7) levels achieved in six rats given 0.3 mL of 10 mg/mL A(1-7) in a preparation containing 0.5 mg/mL captopril, 10 mg/mL LLC, 400 mM Citrate, 150 mM NaCl at pH 3.5. Again, the pharmacokinetic parameters of A(1-7) was determined using a non-compartmental model, where individual pharmacokinetics parameters were also determined. In this analysis, AUCs were determined using values >1,000 ng/mL to provide a perspective of the range of exposure. The mean concentration for each time point was calculated and the PK values for these mean values were estimated. The Tmax was achieved approximately 5 to 10 minutes after administration. Half-lives ranged from 7.97 to 25.6 minutes for this treatment group. Total mean A(1-7) exposure over the observation period was 9,399 ng*min/mL with a range of 1,008 to 26,654 ng*min/mL.

TABLE 5

| | 0.3 ml C, ID [10 mg/ml Angiotensin (1-7), Captopril 5 mg/ml, | | | | | | | |
|---|---|---|---|---|---|---|---|---|
| | 10 mg/ml LLC, 400 mM Citrate pH 3.5, 150 mM NaCl] | | | | | | | |
| | Angiotensin (1-7) (ng/mL) | | | | | | | |
| | | | | | | | Mean | |
| Time Point | | | | | | | PK | |
| (min) | Rat 7 | Rat 8 | Rat 7 | Rat 8 | Rat 7 | Rat 8 | Value | Average |
| 0 | 0.99 | 0.40 | 3.31 | 7.63 | 1.55 | 0.63 | | 2.42 |
| 5 | >1000 | 33.50 | 504.00 | 746.00 | 268.89 | 432.86 | | 619.21 |
| 10 | >1000 | 12.30 | 410.00 | 404.00 | 391.25 | 5.34 | | 423.82 |
| 20 | 466.00 | 8.12 | 70.40 | 178.00 | 63.50 | 41.25 | | 137.88 |
| 40 | 27.20 | 20.20 | 18.40 | 39.90 | 22.70 | 16.10 | | 24.08 |
| 60 | 16.60 | 22.10 | 17.60 | 13.50 | 19.90 | 25.70 | | 19.23 |
| 90 | 10.20 | 0.00 | 11.60 | 5.58 | 2.75 | 4.83 | | 5.83 |
| Cmax (ng/mL) | <1000 | 33.5 | 504 | 746.0 | 391.25 | 432.85 | 639.6 | 619.2 |
| Tmax (ng/mL) | 5.0 | 5.0 | 5.0 | 5.0 | 10 | 5.0 | 5.8 | 5 |
| Half-Life (min) | 10.55 | 7.97 | 15.45 | 11.94 | 12.86 | 25.62 | 14.1 | 13 |
| AUC₀₋₉₀ | 26,654 | 1,008 | 7,641 | 10,668 | 6,228 | 3,862 | 9,343.5 | 9,399 |
| (ng*min/mL) | | | | | | | | |

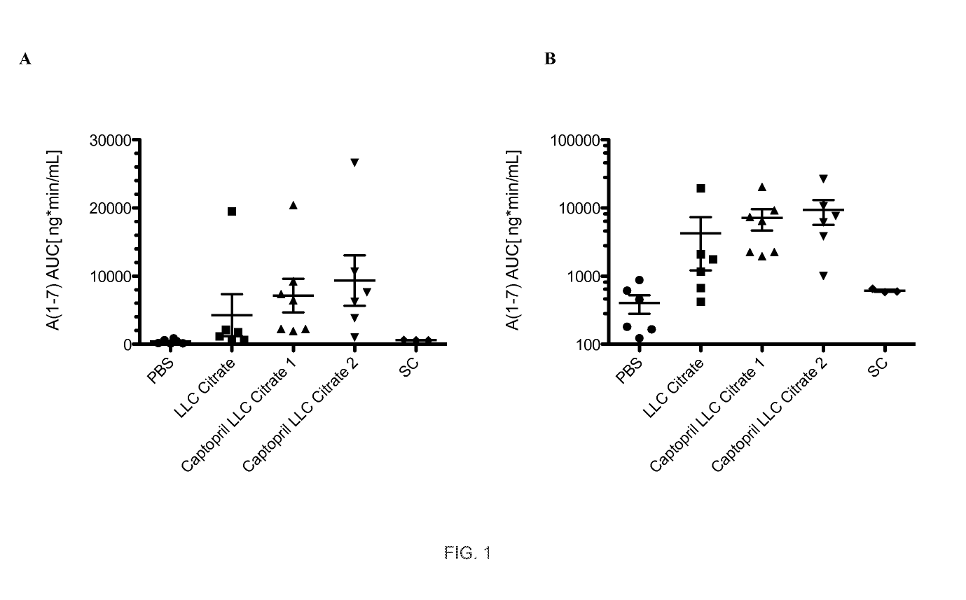

In addition, FIG. 1 illustrates the AUC values compared between various administration routes and formulations.

As shown in Tables 1-5 and FIG. 1, angiotensin (1-7) delivered in a formulation according to the present invention using a rat model mimicking oral delivery has significantly improved half-life and total exposure over the observation period as compared to the baseline profile of angiotensin (1-7) delivered in PBS. These results demonstrate that angiotensin (1-7) can be delivered orally according to the present invention and achieve therapeutically effective bioavailability in circulation.

### EQUIVALENTS AND SCOPE

Those skilled in the art will recognize, or be able to ascertain using no more than routine experimentation, many equivalents to the specific embodiments of the invention described herein. The scope of the present invention is not intended to be limited to the above Description, but rather is as set forth in the following claims:

We claim:

1. A solid dosage form for oral administration comprising
(a) an angiotensin (1-7) peptide;
(b) at least one pharmaceutically acceptable pH-lowering agent;
(c) at least one absorption enhancer effective to promote bioavailability of the angiotensin (1-7) peptide; and
(d) a protective vehicle.

2. The solid dosage form of claim 1, wherein the solid dosage form is a capsule or tablet.

3. The solid dosage form of claim 1 or 2, wherein the pH-lowering agent is citric acid.

4. The solid dosage form of claim 3, wherein the citric acid is present in an amount greater than 400 mg.

5. The solid dosage form of claim 3, wherein the citric acid is present in an amount greater than 500 mg.

6. The solid dosage form of claim 3, wherein the citric acid is present in an amount greater than 50% of the total weight of the solid dosage form.

7. The solid dosage form of claim 1 or 2, wherein the pH-lowering agent is tartaric acid.

8. The solid dosage form of any one of the preceding claims, wherein the absorption enhancer is an acylcarnitine.

9. The solid dosage form of claim 8, wherein the acylcarnitine is lauroyl carnitine.

10. The solid dosage form of claim 9, wherein the lauroyl carnitine is present in an mount ranging from 50-100 mg.

11. The solid dosage form of claim 9, wherein the lauroyl carnitine is present in an mount ranging from 5-10% of the total weight of the solid dosage form.

12. The solid dosage form of any one of the preceding claims, wherein the protective vehicle constitutes less than 20% of the total weight of the solid dosage form.

13. The solid dosage form of any one of the preceding claims, wherein the protective vehicle is an enteric coat.

14. The solid dosage form of any one of the preceding claims, wherein the solid dosage form further comprises one or more excipients selected from fillers such as PROSOLV®, disintegrants such as POLYPLASDONE™ crospovidone, glidants such as silicon dioxide or lubricants such as sodium stearyl fumarate.

15. The solid dosage form of any one of the preceding claims, wherein the solid dosage form further comprises captopril.

16. The solid dosage form of any one of the preceding claims, wherein the solid dosage form has a total weight ranging from 800-1200 mg.

17. The solid dosage form of any one of the preceding claims, wherein the angiotensin (1-7) peptide is present in an amount ranging from 10-1000 mg.

18. A solid dosage form for oral administration comprising
(a) an angiotensin (1-7) peptide;
(b) citric acid;
(c) lauroyl carnitine; and
(d) a protective vehicle.

19. The solid dosage form of claim 18, wherein the citric acid is present in an amount great than 500 mg and the lauroyl carnitine is present in an amount ranging from 50-100 mg.

20. The solid dosage form of claim 18 or 19, wherein the solid dosage form is a capsule or tablet.

21. The solid dosage form of any one of claims 18-20, wherein the protective vehicle is an enteric coat.

22. The solid dosage form of any one of the preceding claims, wherein the angiotensin (1-7) peptide comprises the naturally-occurring Angiotensin (1-7) amino acid sequence of Asp¹-Arg²-Val³-Tyr⁴-Ile⁵-His⁶-Pro⁷ (SEQ ID NO:1).

23. The solid dosage form of any one of claims 1-21, wherein the angiotensin (1-7) peptide is a functional equivalent of SEQ ID NO:1.

24. The solid dosage form of claim 23, wherein the functional equivalent is a linear peptide.

25. The solid dosage form of claim 24, wherein the linear peptide comprises a sequence that includes at least four amino acids from the seven amino acids that appear in the naturally-occurring Angiotensin (1-7), wherein the at least four amino acids maintain their relative positions as they appear in the naturally-occurring Angiotensin (1-7).

26. The solid dosage form of claim 24, wherein the linear peptide comprises a sequence that includes at least five amino acids from the seven amino acids that appear in the naturally-occurring Angiotensin (1-7), wherein the at least five amino acids maintain their relative positions as they appear in the naturally-occurring Angiotensin (1-7).

27. The solid dosage form of claim 24, wherein the linear peptide comprises a sequence that includes at least six amino acids from the seven amino acids that appear in the naturally-occurring Angiotensin (1-7), wherein the at least six amino acids maintain their relative positions as they appear in the naturally-occurring Angiotensin (1-7).

28. The solid dosage form of any one of claim 25-27, wherein the at least four, five or six amino acids, respectively, further maintain their relative spacing as they appear in the naturally-occurring Angiotensin (1-7).

29. The solid dosage form of any one of claims 24-28, wherein the linear peptide contains 4-25 amino acids.

30. The solid dosage form of any one of claims 24-29, wherein the linear peptide is a fragment of the naturally-occurring Angiotensin (1-7).

31. The solid dosage form of any one of claims 24-30, wherein the linear peptide contains amino acid substitutions, deletions and/or insertions in the naturally-occurring Angiotensin (1-7).

32. The solid dosage form of claim 31, wherein the linear peptide has an amino acid sequence of Asp¹-Arg²-Nle³-Tyr⁴-Ile⁵-His⁶-Pro⁷ (SEQ ID NO:2).

33. The solid dosage form of claim 31, wherein the linear peptide has an amino acid sequence of Asp¹-Arg²-Val³-Ser⁴-Ile⁵-His⁶-Cys⁷ (SEQ ID NO:6).

34. The solid dosage form of claim 23, wherein the functional equivalent is a cyclic peptide.

35. The solid dosage form of claim 34, wherein the cyclic peptide comprises a linkage between amino acids.

36. The solid dosage form of claim 35, wherein the linkage is located at residues corresponding to positions Tyr⁴ and Pro⁷ in naturally-occurring Angiotensin (1-7).

37. The solid dosage form of claim 35 or 36, wherein the linkage is a thioether bridge.

38. The solid dosage form of any one of claims 34-37, wherein the cyclic peptide comprises an amino acid sequence otherwise identical to the naturally-occurring Angiotensin (1-7) amino acid sequence of Asp¹-Arg²-Val³-Tyr⁴-Ile⁵-His⁶-Pro⁷ (SEQ ID NO:1).

39. The solid dosage form of any one of claims 34-38, wherein the cyclic peptide comprises a norleucine (Nle) replacing position Val³ in naturally-occurring Angiotensin (1-7).

40. The solid dosage form of claim 36, wherein the cyclic peptide is a 4,7-cyclized angiotensin (1-7) with the following formula:

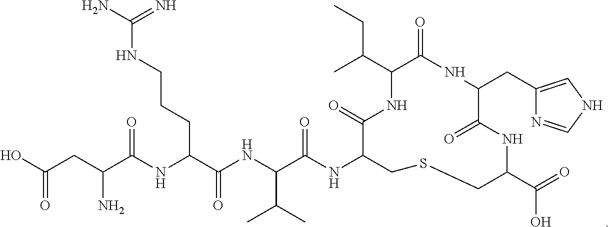 [SMILES: CCC(C)C1NC(=O)C(NC(=O)C(NC(=O)C(CCCNC(=N)N)NC(=O)C(N)CC(=O)O)C(C)C)CSCC(C(=O)O)NC(=O)C(Cc2c[nH]cn2)NC1=O]

41. The solid dosage form of any one of claims 34-40, wherein the angiotensin (1-7) peptide comprises one or more chemical modifications to increase protease resistance, serum stability and/or bioavailability.

42. The solid dosage form of claim 41, wherein the one or more chemical modifications comprise pegylation.
